(19) United States
(12) Patent Application Publication
(10) Pub. No.: US 2023/0391843 A1
(43) Pub. Date: Dec. 7, 2023

(54) COMPOSITION AND METHOD FOR CONTROLLING HEAT GENERATION WITHIN ORGANISM, AND USE

(71) Applicant: GUANGDONG JIANTEBO BIOTECHNOLOGY CO., LTD., Zhongshan (CN)

(72) Inventors: Zhinan YIN, Zhongshan, Guangdong (CN); Qian WANG, Zhongshan, Guangdong (CN); Hengwen YANG, Zhongshan, Guangdong (CN)

(21) Appl. No.: 18/321,062

(22) Filed: May 22, 2023

Related U.S. Application Data
(63) Continuation-in-part of application No. PCT/CN2022/114928, filed on Aug. 25, 2022.

(30) Foreign Application Priority Data
Aug. 26, 2021 (CN) 202110986914.X

(51) Int. Cl. C07K 14/54; A61P 3/00; G01N 33/53

(52) U.S. Cl. CPC C07K 14/54; G01N 2800/52; G01N 33/53; A61P 3/00

(57) ABSTRACT

The present disclosure provides a composition and method used for controlling heat generation in an organism, and a use. The composition of the present disclosure comprises a molecule used for modulating a combination of EBI-3 and p28, or a combination of dimers of the two and receptors thereof, or a downstream signal pathway. The present disclosure discloses the effect of IL27 signals in regulating heat generation. Molecular mechanism studies confirm that the composition acts directly on adipocytes, activates p38 MAPK-PGC1 signals and stimulates the production of UCP1. The therapeutic administration of IL-27 reverses hypothermia induced by the low temperature of EBI-3 KO mice. Therefore, the present disclosure discloses a key effect of IL-27 signals in an energy metabolism process and provides a feasible solution for controlling the body temperature.

### CROSS-REFERENCE TO RELATED APPLICATIONS

The present disclosure claims priority to Chinese Patent Application No. CN202110986914.X, entitled “Composition and Method for Controlling Heat Generation within Organism, and Use”, filed with China National Intellectual Property Administration on Aug. 26, 2021, the entire content of which is incorporated herein by reference.

### TECHNICAL FIELD

The present disclosure relates to the field of biotechnology, and particularly relates to a composition, a method and a use, and particularly to a composition and a method for controlling heat generation within an organism, and a use.

### BACKGROUND ART

Heat generation of brown and beige fat plays a key role in a development of maintenance of a body temperature. Although orientation and activation of the brown/beige fat are known, diversity and abundance of immune factors in adipose tissue are unclear.

### SUMMARY

The present disclosure provides a composition for controlling heat generation within an organism, including a molecule for adjusting binding of EBI-3 and p28 or binding of dimers of EBI-3 and p28 and receptors thereof, or a downstream signal pathway.

Optionally, the adjusting refers to inhibition or reduction of the binding of EBI-3 and p28, or inhibition or reduction of the binding of the dimers of EBI-3 and p28 and the receptors thereof, or down regulation of the downstream signal pathway, and the controlling refers to inhibition of heat generation, thereby reducing a body temperature of the organism.

Optionally, the molecule includes a biological macromolecule or small molecule compound, comprising an anti-EBI-3 antibody, an anti-p28 antibody, an anti-IL-27R antibody, an EBI-3 soluble fragment, a p28 soluble fragment and an IL-27R soluble fragment.

Optionally, the adjusting refers to promotion or enhancement of the binding of EBI-3 and p28, or promotion or enhancement of the binding of the dimers of EBI-3 and p28 and the receptors thereof, or up regulation of the downstream signal pathway, and the controlling refers to promotion of heat generation, thereby improving the body temperature of the organism.

Optionally, the molecule includes a recombinant IL-27 protein, a nuclear acid fragment like mRNA, virus or plasmid DNA that can express IL-27 protein in vivo, a modifier thereof, and polypeptide or a small molecule compound which can activate activity of IL-27R.

The present disclosure provides a method for controlling heat generation within an organism, in which a molecule for adjusting binding of EBI-3 and p28 or binding of dimers of EBI-3 and p28 and receptors thereof, or a downstream signal pathway is applied to the organism.

Optionally, the organism includes a human subject or tissue or cell thereof, a mammal or tissue or cell thereof, or a microorganism.

Optionally, the tissue or cell is in vitro tissue or cell.

The present disclosure further provides a method for screening a candidate compound capable of controlling heat generation within an organism, including a step of measuring a binding condition of EBI-3 and p28, or a binding condition of dimers of EBI-3 and p28 and receptors thereof, or a downstream signal pathway triggered by the binding before and after application of a to-be-tested compound.

Optionally, when the binding of EBI-3 and p28 is inhibited or reduced or the binding of the dimers of EBI-3 and p28 and the receptors thereof is inhibited or reduced or a downstream signal triggered by the binding is down-regulated after the to-be-tested compound is applied, the to-be-tested compound is identified as a candidate compound for inhibiting heat generation within the organism.

Optionally, when the binding of EBI-3 and p28 is promoted or enhanced or the binding of the dimers of EBI-3 and p28 and the receptors thereof is promoted or enhanced or the downstream signal triggered by the binding is up-regulated after the to-be-tested compound is applied, the to-be-tested compound is identified as a candidate compound for promoting heat generation within the organism.

Optionally, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in an organism model, so as to obtain a first measured value;
b. after the to-be-tested compound is applied to the organism model, measuring the parameter of the binding of EBI-3 and p28 or the parameter of the binding of the dimers of EBI-3 and p28 and the receptors thereof, or the parameter of the activation condition of the downstream signal pathway in the organism model, so as to obtain a second measured value; and
c. comparing the first measured value and the second measured value.

Optionally, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in a first organism model, so as to obtain a first measured value;
b. measuring a parameter of the binding of EBI-3 and p28 or a parameter of the binding of the dimers of EBI-3 and p28 and the receptors thereof, or a parameter of an activation condition of the downstream signal pathway in a second organism model, so as to obtain a second measured value, wherein the first organism model and the second organism model belong to the same model, the to-be-tested compound is not applied to the first organism model, and the to-be-tested compound is applied to the second organism model; and
c. comparing the first measured value and the second measured value.

Optionally, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in an organism model after the to-be-tested compound is applied, so as to obtain a measured value; and
b. comparing the measured value with a reference value, wherein the reference value is a parameter of the binding of EBI-3 and p28 or a parameter of the binding of the dimers of EBI-3 and p28 and the receptors thereof, or a parameter of an activation condition of the downstream signal pathway measured by the organism model when the to-be-tested compound is not applied.

Optionally, the organism model includes an animal model or a cell model.

The present disclosure further provides use of the composition in any one of the above descriptions in treatment of a metabolic disease.

Optionally, the metabolic disease includes hypothermia.

The present disclosure further provides a method for treating a metabolic disease, in which the composition in any one of the above descriptions is administered to the subject in need.

Optionally, the metabolic disease includes hypothermia.

### BRIEF DESCRIPTION OF DRAWINGS

FIG. 1A-FIG. 1J are diagrams showing promotion of heat generation and energy expenditure by interleukin-27 (IL-27) signals.

FIG. 2A-FIG. 2I are diagrams showing data in which IL-27 directly targets adipocytes to promote heat generation.

FIG. 3A-FIG. 3M are diagrams showing promotion of heat generation by IL-27.

FIG. 4A-FIG. 4J reduction in energy expenditure and heat generation in IL-27 signal-deficient mice.

FIG. 5A-FIG. 5I show that thermogenesis of IL-27Rα signals is not achieved by direct action on CD2+ lymphocytes or Lyz2+ myeloid cells.

FIG. 6A-FIG. 6Q show the phenotype of an IL-27Rα KO and WT chimera in HFD-induced adaptive heat generation.

FIG. 7A-FIG. 7O show that IL-27 up-regulates an uncoupling protein 1 (UCP1) and improves browning of subcutaneous white adipose tissue.

FIG. 8A-FIG. 8AK show that IL-27 promotes heat generation.

### DETAILED DESCRIPTION

Various exemplary embodiments of the disclosure are described now, the detailed description should not be construed as limiting the disclosure but rather as a more detailed description of certain aspects, characteristics and implementations of the disclosure.

It is to be understood that the terminology used herein is for the purpose of describing particular embodiments only and is not intended to be limiting of the disclosure. In addition, for a numerical range in the disclosure, it is to be understood that the upper and lower limits of the range, and each intervening value therebetween, are specifically disclosed. Each smaller range between any stated value or any intervening value in a stated range and any other stated value or any other intervening value in the stated range is encompassed within the disclosure. The upper and lower limits of these smaller ranges may independently be included or excluded in the range.

Unless stated otherwise, all technical and scientific terms used herein have the same meaning as commonly understood by those skilled in the art to which the disclosure belongs. Although only preferred methods and materials are described herein, any methods and materials similar or equivalent to those described herein can be used in the implementation or testing of the disclosure. All documents mentioned in the specification are incorporated by reference to disclose and describe methods and/or materials related to the documents. In case of conflict with any incorporated document, the specification will control. Unless state otherwise, “%” is a percent by weight.

As used herein, the term “heat generation” may be used interchangeably with the terms “heat production”, “heat generation effect” and “thermogenesis”. Thermogenesis used herein means a process in which mammals, including humans, regulate heat generation.

In the present disclosure, binding of an EB virus-induced gene 3 (EBI-3) and a subunit of cytokine IL-27 secreted by dendritic cells (p28) means that an EBI-3 subunit and a p28 subunit form a heterodimeric structure with a certain spatial arrangement by interaction which may be connection by a disulfide bond.

The term “receptor” as used herein refers to a biological macromolecule capable of being bound to the above-mentioned dimers and of causing a change in cell functions. The receptor contains at least an active site which recognizes and is bound to the dimer and a functional active site responsible for generating a response reaction, and thus initiates a series of biochemical reactions, ultimately leading to generation of a biological effect by a target cell. The binding between the receptor and the dimer in the disclosure results in activation of the receptor and creates a basic step of activating subsequent signaling by the receptor. Under physiological conditions, the binding between the receptor and the dimer is not mediated by covalent bonds, but rather is bounded to each other primarily by ionic bonds, hydrogen bonds, Van der Waals forces, and hydrophobic interaction.

The term “signal pathway” as used herein refers to a phenomenon that when a certain reaction occurs in a cell, a signal transmits information from an exterior of the cell to an interior of the cell, and the cell responds according to the information. The signal pathway in the disclosure particularly refers to a series of enzymatic reaction pathways which can transmit extracellular molecular signals into the cell through a cell membrane to exert an effect.

Some embodiments of the present disclosure provide a composition for controlling heat generation within an organism.

The present disclosure provides a composition for controlling heat generation within an organism, including a molecule for adjusting binding of EBI-3 and p28 or binding of dimers of EBI-3 and p28 and receptors thereof, or a downstream signal pathway.

In some embodiments, the adjusting refers to inhibition or reduction of the binding of EBI-3 and p28, or inhibition or reduction of the binding of the dimers of EBI-3 and p28 and the receptors thereof, or down regulation of the downstream signal pathway, and the controlling refers to inhibition of heat generation, thereby reducing a body temperature of the organism.

In some embodiments, the molecule includes a biological macromolecule or small molecule compound, comprising an anti-EBI-3 antibody, an anti-p28 antibody, an anti-IL-27R antibody, an EBI-3 soluble fragment, a p28 soluble fragment and an IL-27R soluble fragment.

In some embodiments, the adjusting refers to promotion or enhancement of the binding of EBI-3 and p28, or promotion or enhancement of the binding of the dimers of EBI-3 and p28 and the receptors thereof, or up regulation of the downstream signal pathway, and the controlling refers to promotion of heat generation, thereby improving the body temperature of the organism.

In some embodiments, the molecule includes a recombinant IL-27 protein, a nuclear acid fragment like mRNA, virus or plasmid DNA that can express IL-27 protein in vivo, a modifier thereof, and polypeptide or a small molecule compound which can activate activity of IL-27R.

Optionally, the composition according to the disclosure includes a molecule for adjusting IL27 signal or binding of IL27 to a receptor thereof or the downstream signal pathway. The downstream signal pathway includes, but is not limited to, JAK/STAT3, p38 MAPK, or the like. In the present disclosure, thermogenesis and energy expenditure are mediated by adipocytes which include, but are not limited to, brown/beige adipocytes and white adipocytes. In the present disclosure, the above-mentioned adipocytes act as IL27 target cells, not immune cells.

Some embodiments of the present disclosure provide a method for controlling heat generation within an organism, in which a molecule for adjusting binding of EBI-3 and p28 or binding of dimers of EBI-3 and p28 and receptors thereof, or a downstream signal pathway is applied to the organism.

In some embodiments, the organism includes a human subject or tissue or cell thereof, a mammal or tissue or cell thereof, or a microorganism.

In some embodiments, the tissue or cell is in vitro tissue or cell.

Some embodiments of the present disclosure provide a method for screening a candidate compound capable of controlling heat generation within an organism.

In the present disclosure, the screening method at least includes a step of measuring, using a reagent, a binding condition of EBI-3 and p28, or a binding condition of dimers of EBI-3 and p28 and receptors thereof, or a downstream signal pathway triggered by the binding before and after application of the to-be-tested compound. The above binding condition is further measured by a step of measuring a relevant parameter using a reagent, wherein the parameter includes a quantity of the dimer formed by EBI-3 and p28, or a metabolic phenotype, or a quantity of a thermogenic protein. The thermogenic protein includes, but is not limited to, UCP1, a peroxisome proliferator-activated receptor (PPARα) and a peroxisome proliferator-activated receptor-γ co-activator-1α (PGC-1α). In the present disclosure, activation (also called as excitation sometimes) of the downstream signal pathway refers to further expression of a downstream target gene. Optionally, the reagent includes a sequence shown by SEQ ID NO: 1-30.

In some embodiments, when the binding of EBI-3 and p28 is inhibited or reduced or the binding of the dimers of EBI-3 and p28 and the receptors thereof is inhibited or reduced or a downstream signal triggered by the binding is down-regulated after the to-be-tested compound is applied, the to-be-tested compound is identified as a candidate compound for inhibiting heat generation within the organism.

In some embodiments, when the binding of EBI-3 and p28 is promoted or enhanced or the binding of the dimers of EBI-3 and p28 and the receptors thereof is promoted or enhanced or the downstream signal triggered by the binding is up-regulated after the to-be-tested compound is applied, the to-be-tested compound is identified as a candidate compound for promoting heat generation within the organism.

In some embodiments, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in an organism model, so as to obtain a first measured value;
b. after the to-be-tested compound is applied to the organism model, measuring the parameter of the binding of EBI-3 and p28 or the parameter of the binding of the dimers of EBI-3 and p28 and the receptors thereof, or the parameter of the activation condition of the downstream signal pathway in the organism model, so as to obtain a second measured value; and
c. comparing the first measured value and the second measured value.

In some embodiments, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in a first organism model, so as to obtain a first measured value;
b. measuring a parameter of the binding of EBI-3 and p28 or a parameter of the binding of the dimers of EBI-3 and p28 and the receptors thereof, or a parameter of an activation condition of the downstream signal pathway in a second organism model, so as to obtain a second measured value, wherein the first organism model and the second organism model belong to the same model, the to-be-tested compound is not applied to the first organism model, and the to-be-tested compound is applied to the second organism model; and
c. comparing the first measured value and the second measured value.

In some embodiments, the method includes the following steps:
a. measuring a parameter of the binding of EBI-3 and p28 or a parameter of the binding of the dimers of EBI-3 and p28 and the receptors thereof, or a parameter of an activation condition of the downstream signal pathway in an organism model after the to-be-tested compound is applied, so as to obtain a measured value; and
b. comparing the measured value with a reference value, wherein the reference value is a parameter of the binding of EBI-3 and p28 or a parameter of the binding of the dimers of EBI-3 and p28 and the receptors thereof, or a parameter of an activation condition of the downstream signal pathway measured by the organism model when the to-be-tested compound is not applied.

In some embodiments, the organism model includes an animal model or a cell model.

The present disclosure further provides use of the composition in any one of the above descriptions in treatment of a metabolic disease.

The present disclosure further provides a method for treating a metabolic disease, in which the composition in any one of the above descriptions is administered to the subject in need.

In some embodiments, the metabolic disease includes hypothermia.

The present disclosure discloses functions of the IL-27(EBI-3/p28) signals in the improvement of in vivo heat generation. It is proved that IL-27 directly acts on the adipocytes, activates the p38 MAPK-PGC1 signals and stimulates generation of UCP1. Therapeutic administration of IL-27 may reverse hypothermia of EBI-3 KO mice induced by the low temperature.

The present disclosure provides a composition and a method for controlling heat generation within an organism, and a use. Research of the present disclosure finds that (EBI-3/p28) signals have an important function in an improvement of in vivo heat generation, and proves that IL-27 directly acts on adipocytes, activates p38 MAPK-PGC1 signals and stimulates generation of UCP1.

### Example

### 1. Experimental Material

### 1.1 Material

### 1.1.1 Mouse

Interleukin-27Rα KO (B6N.129P2-Il27ra^{tmMak}/J, No.: 018078) and EBI-3 KO (B6.129X1-Ebi3^{tm1Rsb}/J, No.: 008691) mice were purchased from Jackson Laboratory. Adipoq-cre mice were supplied by Professor Yong Liu. IL-27Rα^{f/f} mice [herein] were prepared by our own laboratory. Animal experiments were conducted in accordance with ethical specifications and protocols approved by the institutional animal protection and utilization committee of Jinan University and the institutional animal care and utilization committee of Yale University. Groups of mice with matched genders and ages were used in all experiments, with 4 to 6 animals per cage. IL-27 treatment experiments and bone marrow chimera production were performed in random groups. The groups of mice were bred in a specific sterile animal facility with a controlled temperature and humidity, the temperature was 25° C., a cycle of light and dark was 12 h:12 h, and food and water were freely obtained.

### 1.1.2 Reagent

Human serum samples were analyzed on a Bio-Plex system (BioRad) using a Bio-Plex-Pro™ Human Cytokine 17-Plex analysis kit (Cat. No. M5000031YV) and a Bio-Plex Pro™ Human Inflammation Panel 1 (Cat. No. 171-AL001M). A human IL-27 ELISA kit (434607) was purchased from Biolegend. Glucose (63005518) was purchased from Chinese Sinopharm Chemical Reagent Company. Blood glucose test strips (one-touch) were purchased from Johnson and Johnson. An anti-mouse pAKT monoclonal antibody (4060), an anti-mouse AKT monoclonal antibody (4685), an anti-mouse GAPDH monoclonal antibody (5174), an anti-mouse HSP90 monoclonal antibody (4877), an anti-mouse PPARγ monoclonal antibody (2435), an anti-mouse pSTAT3 monoclonal antibody (9145), an anti-mouse STAT3 monoclonal antibody (9139), an anti-mouse p-p38 MAPK (9211), an anti-mouse p38 MAPK (9212), an anti-mouse pATF2 monoclonal antibody (9221), and an anti-mouse ATF2 monoclonal antibody (35031) were purchased from Cell Signaling Technology. An anti-mouse UCP1 monoclonal antibody (ab10983), an anti-mouse PGC-1α monoclonal antibody (ab54481), an anti-mouse PPARα monoclonal antibody (ab8934), an anti-mouse PRDM16 monoclonal antibody (ab202344) and an anti-mouse IL-27Rα monoclonal antibody (ab5997) were purchased from Abcam. APC-CY7-anti-mouse CD45 (clone 30-F11) was purchased from BD. APC anti-mouse CD11b (clone M1/70) and PE anti-mouse F4/80 (clone BM8) were purchased from Sungene Biotech (Tianjin, China). Recombinant mice IL-27 (rmIL-27) (577408) were purchased from Biolegend. An STAT3 inhibitor SH-4-54 and a p38 MAPK inhibitor SB203580 were purchased from Selleck Chemicals. Brown/beige fat differentiation agent dexamethasone (D4902), rosiglitazone (R2408), indomethacin (I7378), isoproterenol (I5627), forskolin (F6886), 3-isobutyl-1-methylxanthine (IBMX) (I5879), a triglyceride detection kit (T2449) and insulin (I3536) were purchased from Sigma. A leptin detection kit (MOB00) and an adiponectin detection kit (MRP300) were purchased from R and D. An insulin detection kit (90080) was purchased from Crystal Chem.

### 2. Experimental Method

### 2.1 Enzyme-Linked Immunosorbent Assay

Human serum was obtained as described above. Mouse serum was collected after high fat diet (HFD) feeding for 10 weeks. Human IL-27 and mouse insulin were detected using the ELISA kit according to an operation instruction.

### 2.2 Luminex Immunoassay

Human serum was collected as described above. Detection of human serum inflammatory factors was performed on the Bio-Plex 200 flow liquid chip analysis system (BioRad) using the Bio-Plex-Pro™ Human Cytokine 17-Plex analysis kit (Cat. No. M5000031YV) and the Bio-Plex Pro™ Human Inflammation Panel 1 (Cat. No. 171-AL001M) according to instructions of the user manual.

### 2.3 Western Blot

A whole cell lysate or tissue lysate was extracted using an RIPA lysis buffer (Beyotime, China) supplemented with a complete protease inhibitor (Roche) and a phosphatase inhibitor Cocktail 2 (P5726, Sigma), and a supernatant was used for subsequent analysis. Proteins were diluted in a Loading dye (BL502A, Bio-sharp), heated at 95° C. for 10 min, and separated on 4-12% polyacrylamide gel. The proteins were transferred to polyvinylidene fluoride membranes and western blot experiments were performed with the commercial antibodies and methods-reagents mentioned in the drawing.

### 2.4 Histology

Adipose or liver tissue was fixed in 4% paraformaldehyde/1×PBS overnight at 4° C. and embedded in paraffin before sectioning. Sections were stained with hematoxylin and eosin (H and E) or oil red O (liver) and photographed under a bright field microscope.

### 2.5 Immunohistochemistry

UCP1 and immunohistochemistry were performed using a rabbit specific HRP/DAB (ABC) detection IHC kit (ab64261, Abcam) according to instructions of the manufacturer.

### 2.6 Core Body Temperature Measurement and Cold Stimulation Experiment

Cold stimulation experiments were performed in a temperature controlled cold room. Mice were placed individually in individual cages (without bedding) at 4° C. The mice had free access to food and water. Rectal core body temperatures were recorded every 2 hours using a digital thermometer and a rectal thermocouple probe (TH-212, HICHANCE, China). If the core body temperature fell below 20° C., the individual mice were euthanized and scored as events for survival analysis.

### 2.7 Metabolic Cage

Energy loss and energy expenditure of the mice were measured using a comprehensive laboratory animal monitoring system (CLAMS, Columbus Instruments, Columbus, OH) metabolic cage in the environmental control rodent incubator of Yale University. The mice were bred individually and acclimated in metabolic chambers for 48 hours prior to data collection. The mice had free access to food and water. Each mouse was subjected to continuous monitoring of physical activity and food intake. During the experiment, concentrations of CO₂/O₂ were collected 4 times per hour for each mouse.

### 2.8 RNA Extraction and Gene Expression Analysis

Total RNA was extracted from frozen tissue using a TRNzol general reagent (Tiangen, Beijing) and quantified using a Nanodrop 2000 ultraviolet-visible spectrophotometer (Thermo). The cDNA was prepared by 1 μg of total RNA through a reverse transcription polymerase chain reaction using a PrimeScript™ RT Reagent kit (TAKARA, Dalian). Real-time quantitative PCR of the cDNA was performed on a CFX96™ real-time PCR detection system (Bio-Rad) using a TB Green™ Premix-EX-Taq™ II kit (TAKARA). A fold change in expression was calculated by a ΔΔCₜ method using immunohistochemistry with mouse HPRT as an internal reference. Primer pair sequences were as follows.

| | Mouse HPRT: |
|---|---|
| | forward- |
| (SEQ ID NO. 1) | |
| | TCATTATGCCGAGGATTTG |
| | and |
| | reverse- |
| (SEQ ID NO. 2) | |
| | GCCTCCCATCTCCTTCAT; |
| | TNF-α: |
| | forward- |
| | CTACTGAACTTCGGGGTGAT |
| (SEQ ID NO. 3) | |
| | and |
| | reverse- |
| (SEQ ID NO. 4) | |
| | CAGGCTTGTCACTCGAATT; |
| | IL-6: |
| | forward- |
| (SEQ ID NO. 5) | |
| | TCCAGTTGCCTTCTTGGGAC, |
| | and |
| | reverse- |
| (SEQ ID NO. 6) | |
| | GTGTAATTAAGCGCCGACTTG; |
| | IL-12p40: |
| | forward- |
| | GGCTGGTGCAAAGAAACATGGACTTGA, |
| (SEQ ID NO. 7) | |
| | and |
| | reverse- |
| (SEQ ID NO. 8) | |
| | TGCAGACAGAGACGCCATTCCACAT; |
| | IL-1β: |
| | forward- |
| (SEQ ID NO. 9) | |
| | CAACCAACAAGTGATATTCTCCATG, |
| | and |
| | reverse- |
| (SEQ ID NO. 10) | |
| | GATCCACACTCTCCAGCTGCA; |
| | IL-4: |
| | forward- |
| (SEQ ID NO. 11) | |
| | GAAAACTCCATGCTTGAAGAA, |
| | and |
| | reverse- |
| (SEQ ID NO. 12) | |
| | TCTTTCAGTGATGTGGACTTG; |
| | IL-5: |
| | forward- |
| (SEQ ID NO. 13) | |
| | TCACCGAGCTCTGTTGACAA, |
| | and |
| | reverse- |
| (SEQ ID NO. 14) | |
| | CCACACTTCTCTTTTTGGCG; |
| | IL-13: |
| | forward- |
| (SEQ ID NO. 15) | |
| | TGAGCAACATCACACAAGACC, |
| | and |
| | reverse- |
| (SEQ ID NO. 16) | |
| | GGCCTTGCGGTTACAGAGG; |
| | Ucp1: |
| | forward- |
| (SEQ ID NO. 17) | |
| | ACTGCCACACCTCCAGTCATT, |
| | and |
| | reverse- |
| (SEQ ID NO. 18) | |
| | CTTTGCCTCACTCAGGATTGG; |
| | Cox8b: |
| | forward- |
| (SEQ ID NO. 19) | |
| | GAACCATGAAGCCAACGACT, |
| | and |
| | reverse- |
| (SEQ ID NO. 20) | |
| | GCGAAGTTCACAGTGGTTCC; |
| | Cidea: |
| | forward- |
| (SEQ ID NO. 21) | |
| | TGCTCTTCTGTATCGCCCAGT, |
| | and |
| | reverse- |
| (SEQ ID NO. 22) | |
| | GCCGTGTTAAGGAATCTGCTG; |
| | Prdm16: |
| | forward- |
| (SEQ ID NO. 23) | |
| | CAGCACGGTGAAGCCATTC, |
| | and |
| | reverse- |
| (SEQ ID NO. 24) | |
| | GCGTGCATCCGCTTGTG; |
| | Adiponectin: |
| | forward- |
| (SEQ ID NO. 25) | |
| | GAATCATTATGACGGCAGCA, |
| | and |
| | reverse- |
| (SEQ ID NO. 26) | |
| | TCATGTACACCGTGATGTGGTA; |
| | and |
| | Elovl3: |
| | forward- |
| (SEQ ID NO. 27) | |
| | TCCGCGTTCTCATGTAGGTCT, |
| | and |
| | reverse- |
| (SEQ ID NO. 28) | |
| | GGACCTGATGCAACCCTATGA. |

### 2.9 Bone Marrow Chimera

IL-27Rα KO and WT mice which were 10 weeks old were used as bone marrow cell donors. Bone marrow was separated from hind limb femurs, erythrocytes were lysed, a product was filtered by a cell filter (40 μm), and cell pellets were washed 3 times with sterile PBS, counted and stored on ice for injection. The IL-27Rα KO and WT mice which were 10 weeks old were lethally irradiated (900 rads), 1×10⁷ bone marrow cells were transplanted by ocular intravenous injection, the mice were placed in specific pathogen-free facilities and supplemented with sterile water and feed for 8 weeks to reconstitute immune systems, and then, metabolic research was performed.

### 2.10 Primary Beige Adipocyte Preparation

Inguinal subcutaneous adipose tissue was minced and digested at 37° C. for 45 min in PBS containing collagenase II (1 mg/ml), and a tissue suspension was filtered by a 100 μm cell filter and centrifuged at 600 g for 5 min, such that a stromal vascular fraction (SVF) was granulated. After granulation, the product was further filtered by a 40 μm cell filter and placed on a collagen-coated plate. After overnight culturing, a supernatant containing non-adherent cells was removed. Precursor adipocytes were grown in DMEM containing 10% FBS and insulin (5 μg/ml) for fusion. The product was treated for 2 days with dexamethasone (1 μM), 3-isobutyl-1-methylxanthine (IBMX, 0.5 mM), insulin (5 μg/ml), indomethacin (125 nM) and rosiglitazone (1 μM) to induce differentiation of the fused cells, and then independently treated for another 5 days with insulin (5 μg/ml) and triiodothyronine (T3, 1 nM). On day 7, the cells were pretreated overnight with and without IL-27 (100 ng/ml) and then treated with isoproterenol (10 μM) or forskolin (10 μM) for 4-6 hours. IL-27 (100 ng/ml) was added for treatment for 0-120 min on day 7 for protein phosphorylation analysis, or for 12-24 h for protein expression level detection. For signal inhibition experiments, either the STAT3 inhibitor (C188-9, 10 μM) or the p38 MAPK inhibitor (SB203580, 10 μM) was added to a medium 0.5 h prior to and during IL-27 treatment.

### 2.11 Flow Cytometric Analysis (FACS)

Epididymal adipose tissue was minced and digested as described above. SVF particles were used for the surface staining of the APC-CY7 anti-mouse CD45, APC anti-mouse CD11b, PE anti-mouse F4/80, the product was incubated for 15 min, and then the cells were washed with PBS and analyzed using a BD-FACSVerse flow cytometer (BD).

### 2.12 Production of IL-27Rα Conditional Knockout Mouse.

A development of IL-27Rα conditional knockout mice was performed using a gene targeting technology. Briefly, a 997 bps CKO region containing exon 3 and exon 4 of the IL27ra gene (GenBank NM_016671.3), a 5.2 kb 5′ homology arm and a 3 kb 3′ homology arm were amplified from BAC clones using a high fidelity Taq technology, and the resultant, a recombination site and a selectable marker were assembled into a targeting vector. The final targeting vector was determined by multiple times of digestion by restriction endonuclease and full sequence analysis and transformed into 129 s mouse embryonic stem cells by electroporation. A correct embryonic stem cell clone was identified and injected into a C57BL/6J mouse blastocyst. After a chimera mouse was unambiguously identified, the chimera mouse and a C57BL/6J mouse were backcrossed. Genotyping PCR confirmed germ line transmission. Then, mice with an IL-27Rα f/w genotype and B6 adipoq-cre mice were bred. Mice with a desired genotype were selected for the experiment. Genotyping primers at flox or wt were detected:

| | forward: |
|---|---|
| (SEQ ID NO. 29) | |
| | CTGGTTCTGGTATGGTTTGGGGTT |
| | and |
| | reverse: |
| (SEQ ID NO. 30) | |
| | TGAAAGAACTCAACAGTGGGCCGG |

### 2.13 IL-27 Treatment

8-week-old WT mice were fed with HFD for 32 weeks and randomly divided into 2 groups. The mice were injected intraperitoneally with rmIL-27 (100 μg/kg) or PBS for 15 consecutive days, and then, metabolic analysis was performed. 12-month-old EBI-3 KO mice were randomly divided into 2 groups, and injected intraperitoneally with rmIL-27 (100 μg/kg) or PBS for 7 consecutive days. The mice were then subjected to a cold stimulation experiment at 4° C.

### 2.14 Statistical Analysis

Mapping and statistical analysis were performed using Graphpad Prism software (version 7). All statistical tests were fully described in the graph legend and met the criteria for normal distribution with similar variances. A sample size was not predetermined using a statistical method. Comparison between the two groups was performed using a t-test. For evaluation data of relevant samples, repeated measurement analysis of variance (AVOVA) was performed. For an evaluation of more than two groups, one-way analysis of variance and multiple comparison were used. For an evaluation between two independent variables, a two-way analysis of variance with multiple comparison was used. Survival analysis was tested using a log rank. Linear regression analysis was adopted for correlation analysis. The data was expressed by a mean±s.e.m., unless stated otherwise. P<0.05 was considered to be statistically significant, and denoted as *p<0.05, **p<0.01, **p<0.001, and NS meant “not significant”.

### 3. Experiment Result

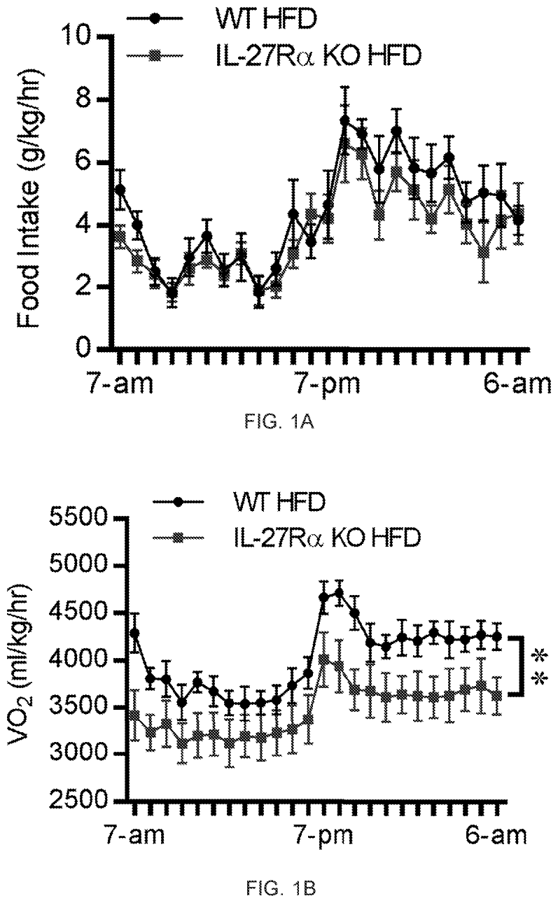

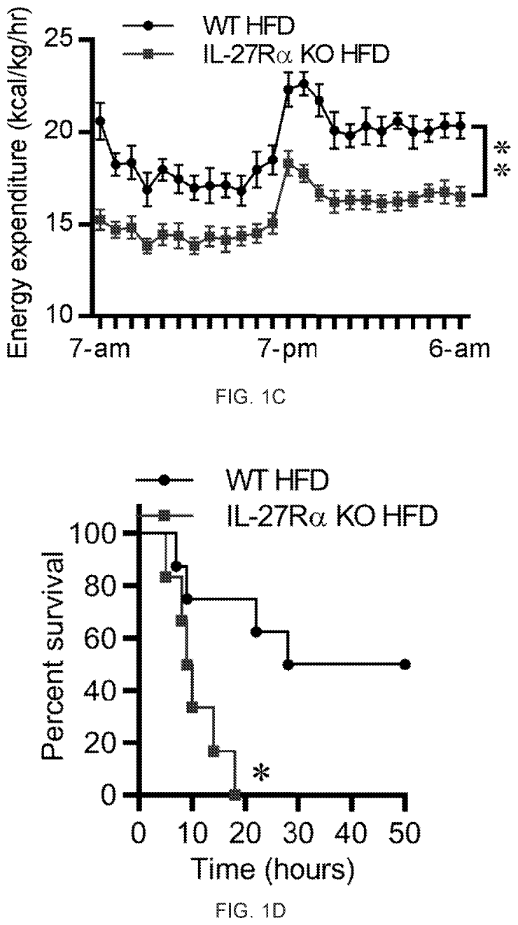

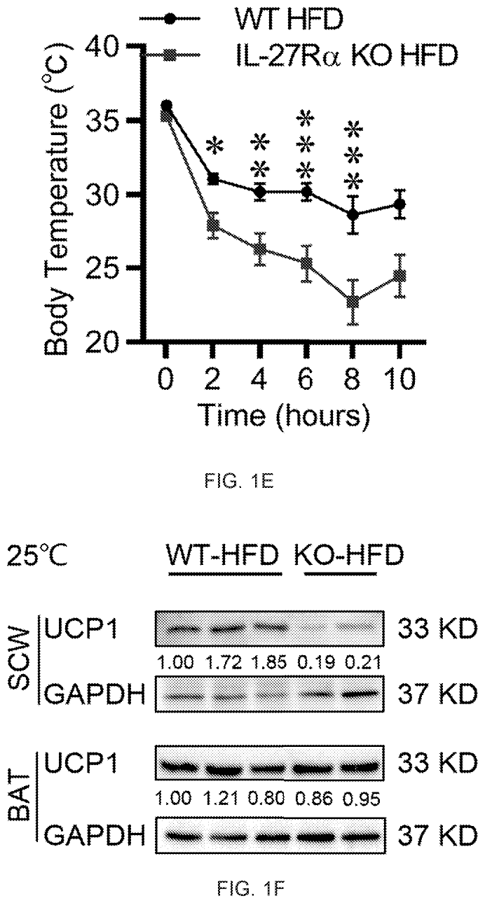

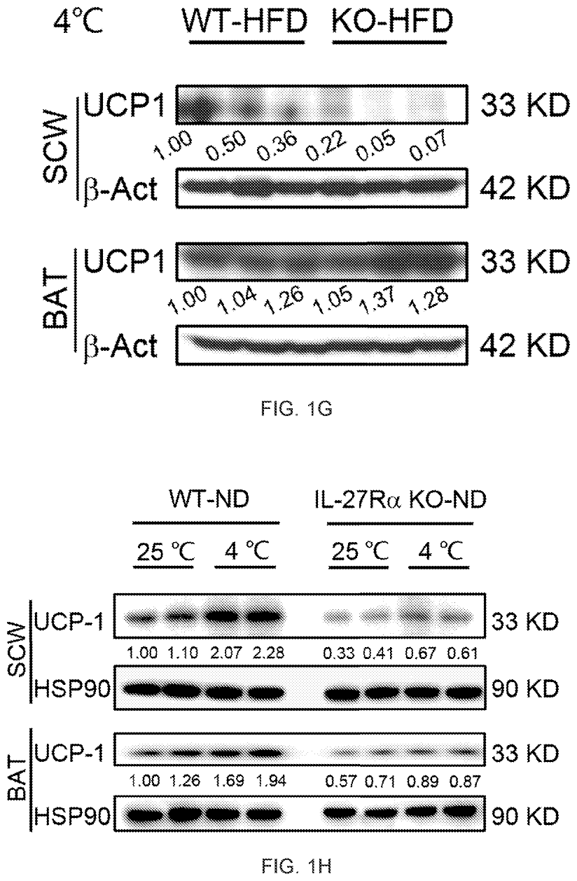

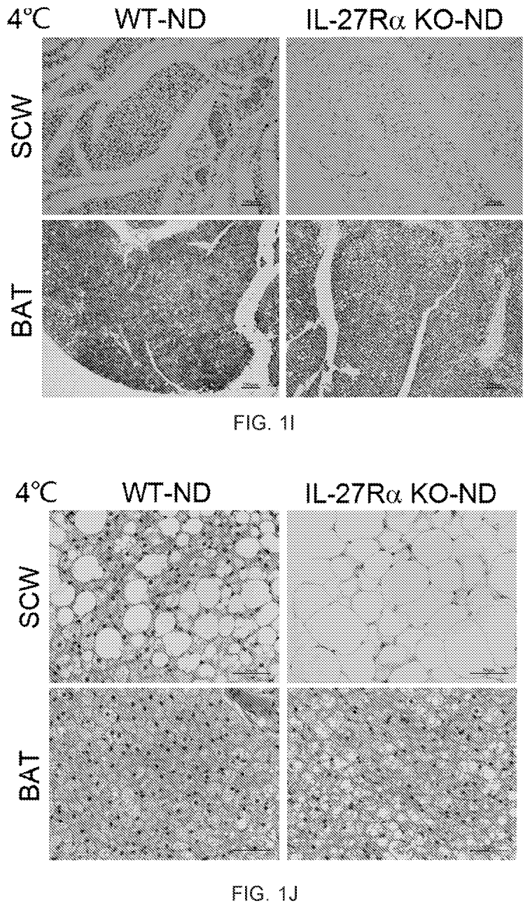

FIG. 1A-FIG. 1J are diagrams of promotion of heat generation and energy expenditure by IL-27 signals. As for FIG. 1A-FIG. 1C: IL-27Rα KO mice and WT control mice were fed with HFD for 4 weeks and then bred in metabolic cages. Food intake (shown in FIG. 1A in the drawing), oxygen consumption (shown in FIG. 1B in the drawing) and energy expenditure (shown in FIG. 1C in the drawing) were monitored in 24 hours (n=7-8). As for FIG. 1D and FIG. 1E: IL-27Rα KO mice and WT control mice were fed with HFD for 6 weeks and then subjected to cold stimulation (4° C.). Survival curves were shown in the drawing (FIG. 1D, n=6-8), and rectal temperatures were shown in the drawing (FIG. 1E, n=11-15). As for FIG. 1F and FIG. 1G: IL-27Rα KO and WT mice were fed with HFD for 6 weeks, and then stayed at 25° C. (shown in FIG. 1F in the drawing) or were subjected to cold stimulation at 4° C. (shown in FIG. 1G in the drawing) for 2 hours. Subcutaneous fat (SCW) and brown adipose tissue (BAT) were collected for UCP1 immunoblot analysis. As for FIG. 1H-FIG. 1J: IL-27Rα KO and WT mice fed with a normal diet stayed at 25° C. or were subjected to cold stimulation at 4° C. for 48 hours. SCW and BAT were collected for UCP1 immunoblot analysis (shown in FIG. 1H in the drawing), FIG. 1I was a UCP1 immunohistochemical staining result (scale=100 μm), and FIG. 1J was H and E staining of BAT and SCW tissue (scale=50 μm). As for (FIG. 1F-FIG. 1H): a band density was quantified and normalized with ImageJ. Each lane represented a biologically independent sample, and the experiment was repeated at least twice with similar results. Data was a mean of the biologically independent samples ±s.e.m., and two-way analysis of variance (FIG. 1A-FIG. 1C and FIG. 1E) was shown; and a log rank test (FIG. 1D) was shown. *p<0.05, **p<0.01, and ***p<0.001.

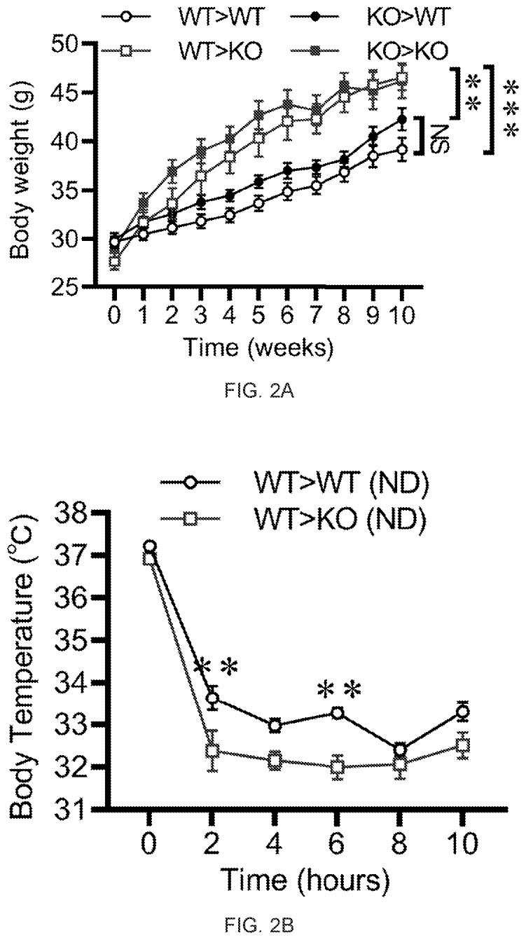

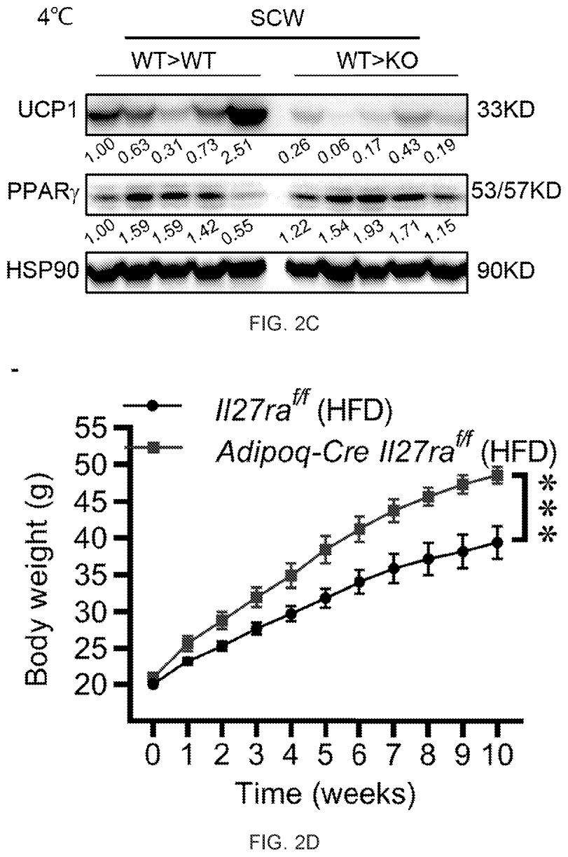

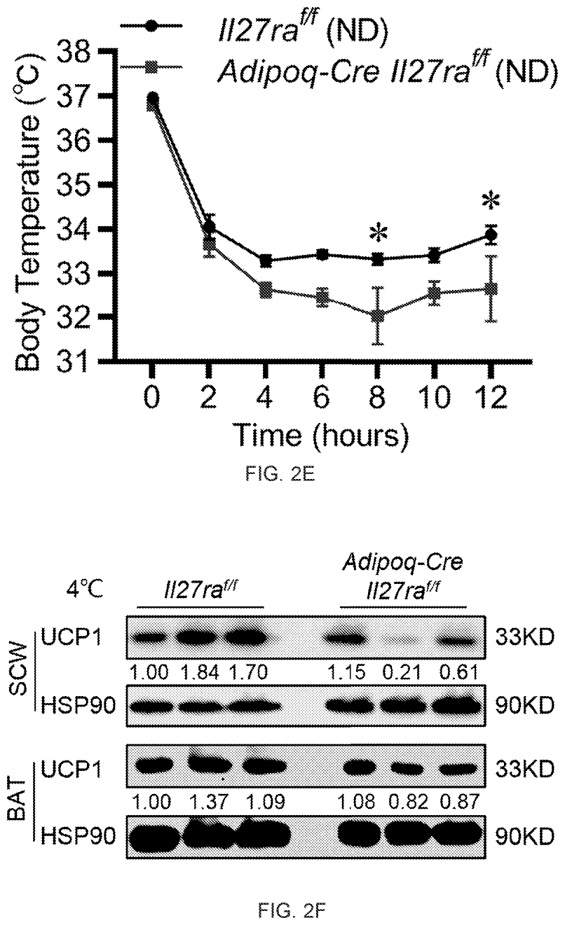

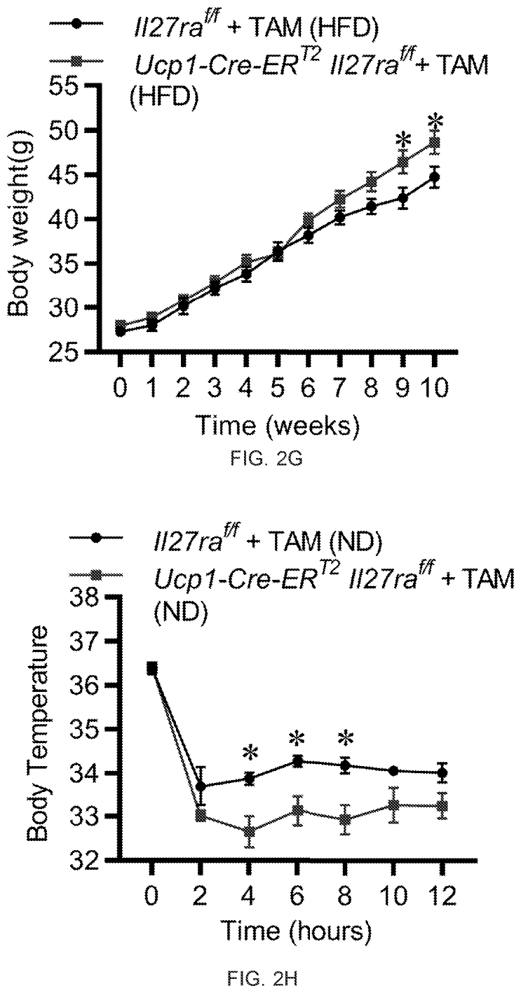

FIG. 2A-FIG. 2I are diagrams in which IL-27 directly targets adipocytes to promote heat generation. As for FIG. 2A: IL-27Rα KO or WT mice were fed with HFD for 10 weeks as donors or receptors to generate four groups of bone marrow chimeras. Body weight was recorded weekly (n=10-13). As for FIG. 2B: an influence of cold stimulation on rectal temperatures of chimera mice fed with a normal diet (4° C., n=6-7) was shown. As for FIG. 2C: after the chimera mice fed with the normal diet were subjected to cold stimulation at 4° C. for 12 hours, SCW was collected for an immunoblot. As for FIG. 2D, 8-week-old Adipoq-Cre Il27ra^{f/f} mice and an Il27ra^{f/f} control group were fed with HFD for 10 weeks. Body weight was recorded weekly (n=7-9). As for FIG. 2E: an influence of cold stimulation on rectal temperatures of mice fed with a normal diet (4° C., n=16-17) was shown. As for FIG. 2F: after the Adipoq-Cre Il27ra^{f/f} and Il27ra^{f/f} mice fed with the normal diet were subjected to cold stimulation at 4° C. for 12 hours, SCW and BAT were collected for an immunoblot. As for FIG. 2G, 8-week-old Ucp1-Cre Il27ra^{f/f} mice and an Il27ra^{f/f} control group were fed with HFD for 10 weeks. Body weight was recorded weekly (n=5-6). As for FIG. 2H: an influence of cold stimulation on rectal temperatures of mice fed with a normal diet (4° C., n=5-8) was shown. As for FIG. 2I: after the Ucp1-Cre Il27ra^{f/f} and Il27ra^{f/f} mice fed with the normal diet were subjected to cold stimulation at 4° C. for 12 hours, SCW and BAT were collected for an immunoblot (FIG. 2C, FIG. 2F and FIG. 2I), and a band density was quantified and normalized with ImageJ. Each lane represented an independent biological sample, and the experiment was repeated at least twice with similar results. Data was a mean of the biologically independent samples ±s.e.m. Two-way analysis of variance (FIG. 2A, FIG. 2B, FIG. 2D, FIG. 2E, FIG. 2G and FIG. 2H) was shown. *p<0.05, **p<0.01, and **p<0.001.

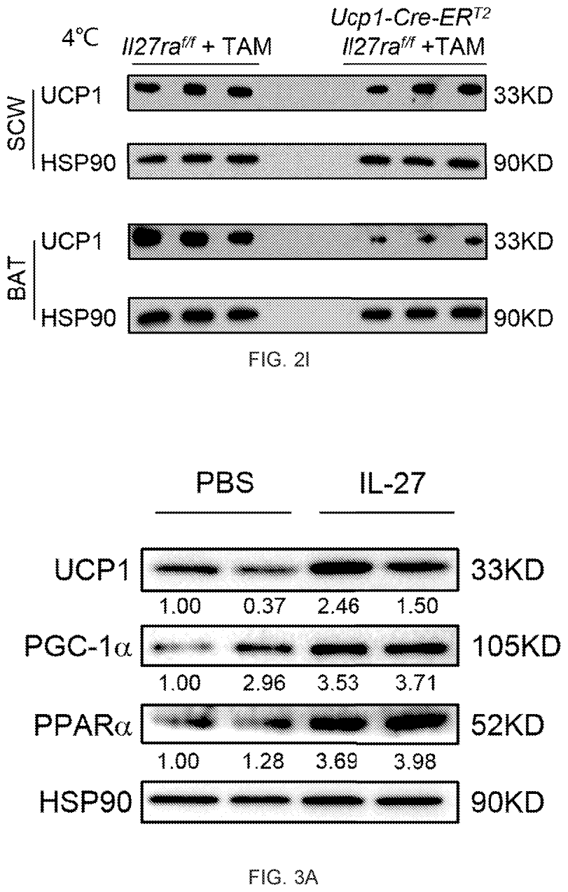

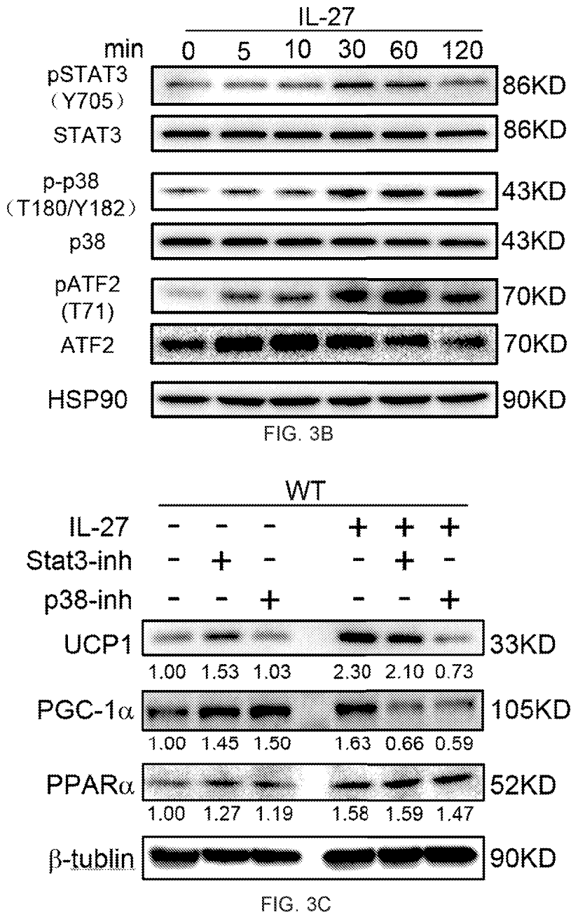

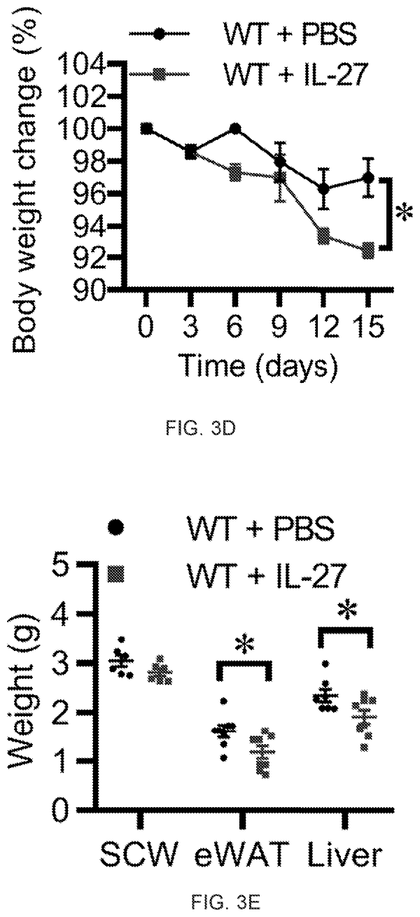

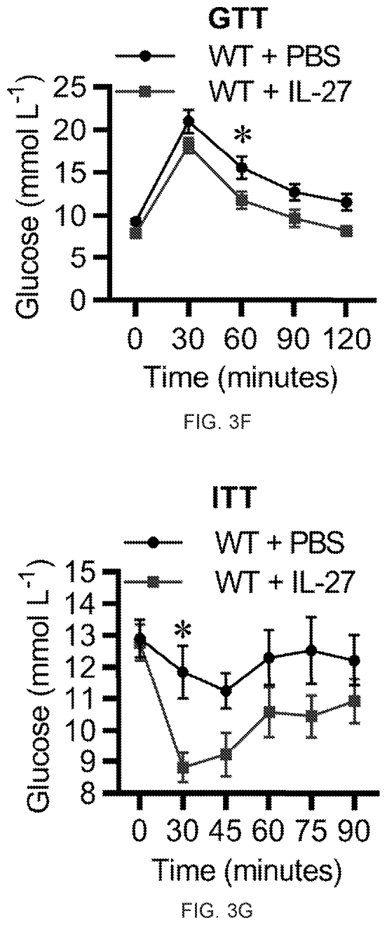

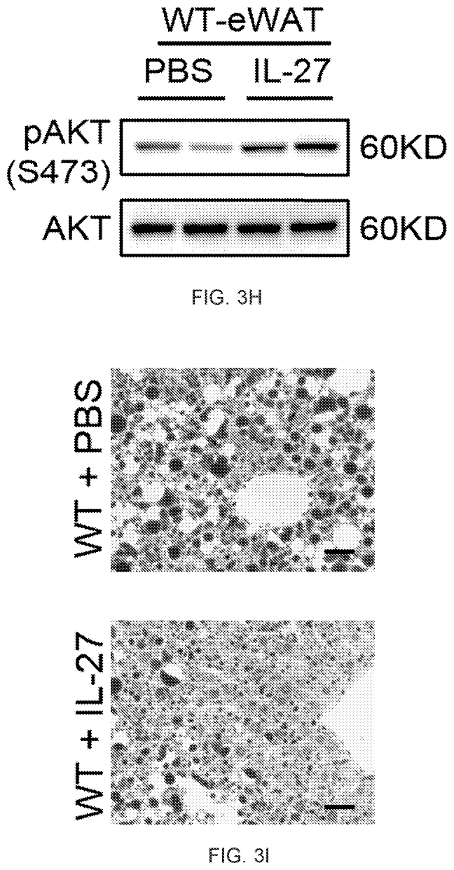

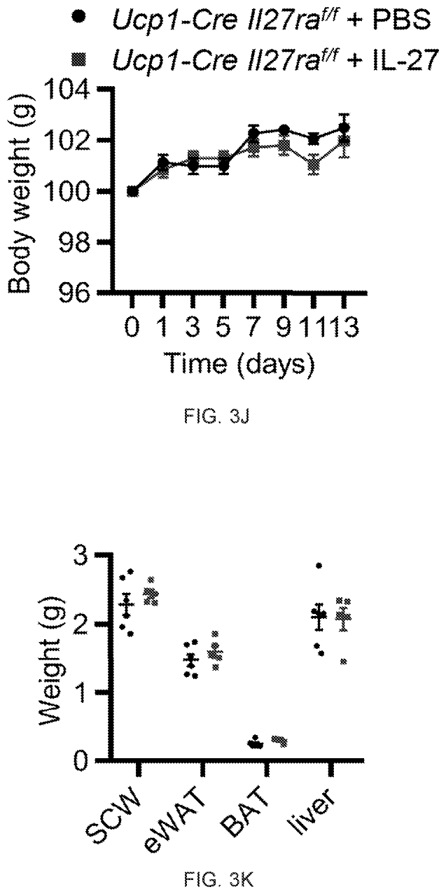

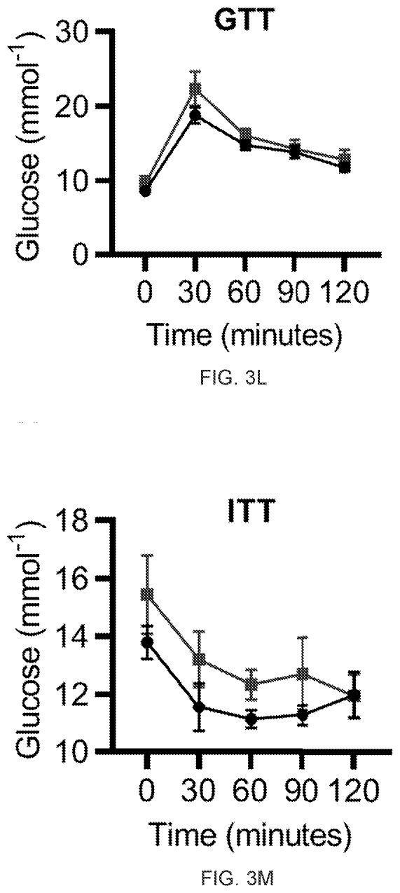

FIG. 3A-FIG. 3M show promotion of heat generation by IL-27. As for FIG. 3A: primary beige adipocytes were generated in vitro from an SVF of WT SCW and then treated with rmIL-27 (100 ng/ml) or PBS for 24 hours. Cells were lysed and a protein extract was used for immunoblot analysis. As for FIG. 3B: immunoblot analysis of phosphorylation of the protein in the WT primary beige adipocyte extract treated with the rmIL-27 (100 ng/ml) for a specified time was shown. As for FIG. 3C: the primary beige adipocytes derived from WT SCW were generated in vitro. The STAT3 inhibitor (C188-9, 10 μM) or p38 MAPK inhibitor (SB203580, 10 μM) was added to a medium 0.5 hours prior to culturing and in duration of rmIL-27 treatment (100 ng/ml for 12 hours). Cell lysis and the representative immunoblot of the protein extract were shown. As for FIG. 3D-FIG. 3J: WT mice were fed with HFD for 32 weeks and then injected intraperitoneally. Every other day, rmIL-27 (100 μg/kg) or PBS was injected for 15 days. As for FIG. 3D: body weight was recorded at specified time points (n=8). As for FIG. 3E: after rmIL-27 treatment for 15 days, adipose tissue was collected and weighed (n=6-8) for a glucose tolerance test (FIG. 3F, n=8) and an insulin tolerance test (FIG. 3G, n=8). As for FIG. 3H: the mice were fasted overnight and then injected intraperitoneally with 0.75 U/kg insulin (15 min). Immunoblot analysis of pAKT (pSer473) in epididymal fat was performed. As for FIG. 3I: after rmIL-27 treatment for 15 days, oil-red O liver tissue staining was performed, with the scale=50 μm. As for FIG. 3J-FIG. 3M: UCP1-KO mice were fed with HFD for 20 weeks and then injected intraperitoneally with rmIL-27 (100 μg/kg) or PBS every other day for 15 days. As for FIG. 3J: body weight was recorded at specified time points (n=6). As for FIG. 3K: adipose tissue and livers were collected and weighed after rmIL-27 treatment for 15 days (n=6), and GTT (FIG. 3L, n=6) and ITT (FIG. 3M, n=4) tests were performed after rmIL-27 treatment for 15 days. A band density was quantified and normalized with ImageJ (FIG. 3A and FIG. 3C), and the experiment was repeated at least twice with similar results. Data was a mean of biologically independent samples ±s.e.m. An unpaired student's t-test was shown (FIG. 3E and FIG. 3K); and two-way analysis of variance was shown (FIG. 3D, FIG. 3F, FIG. 3G, FIG. 3J, FIG. 3L and FIG. 3M). *p<0.05.

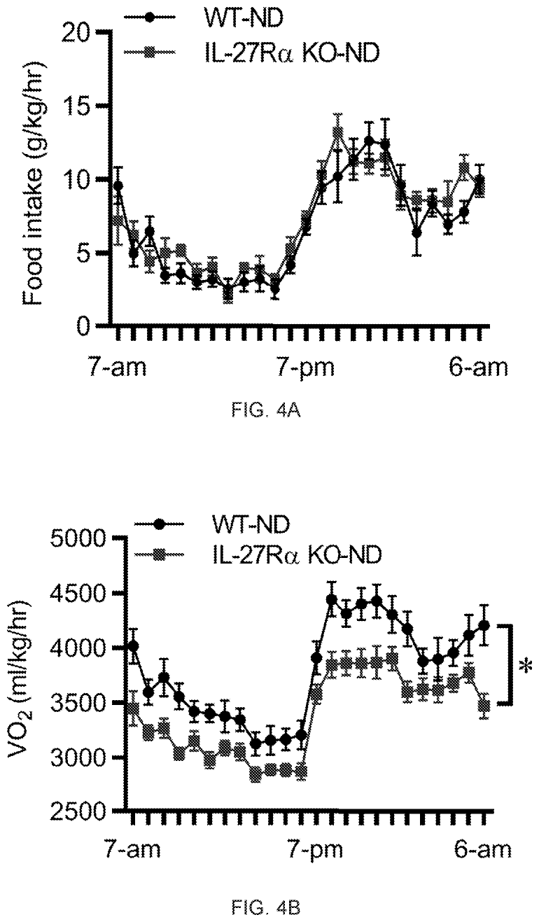

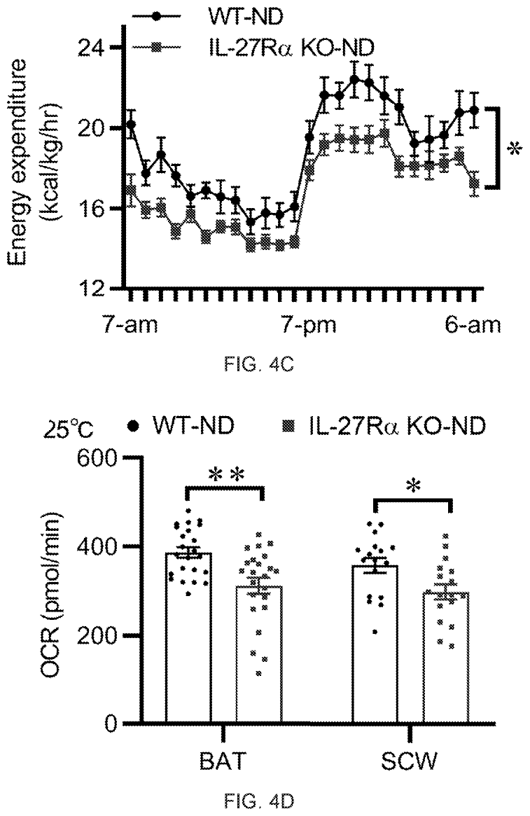

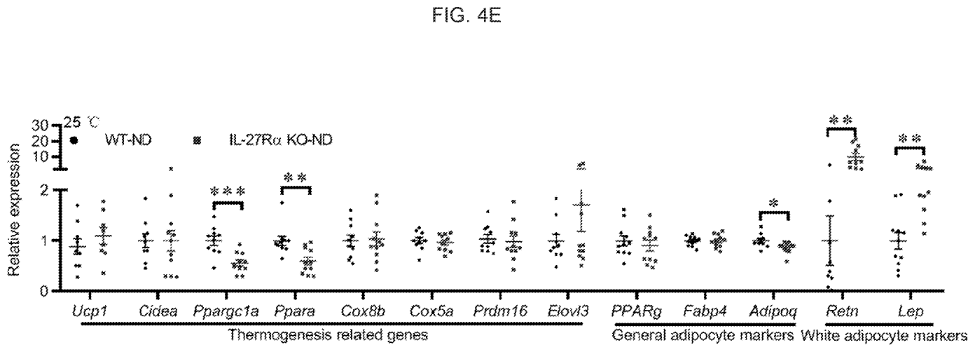

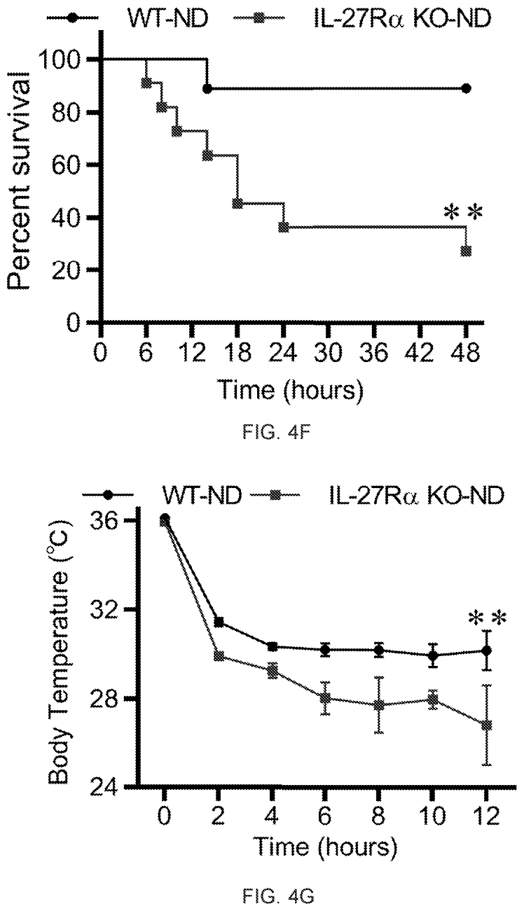

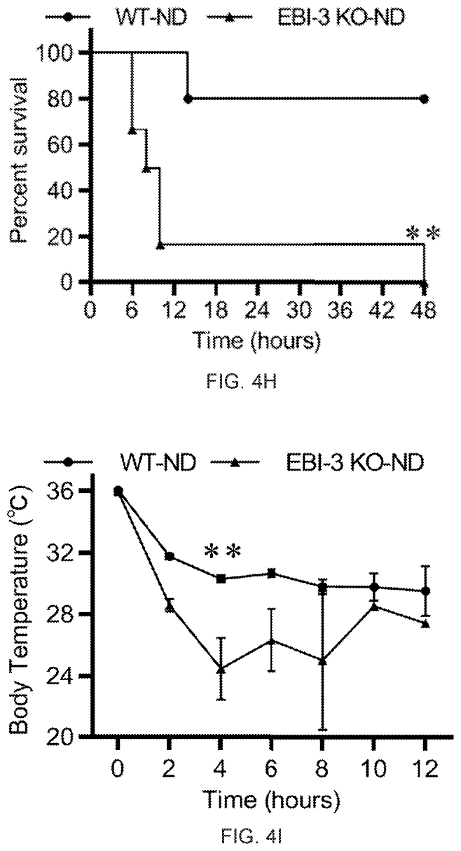

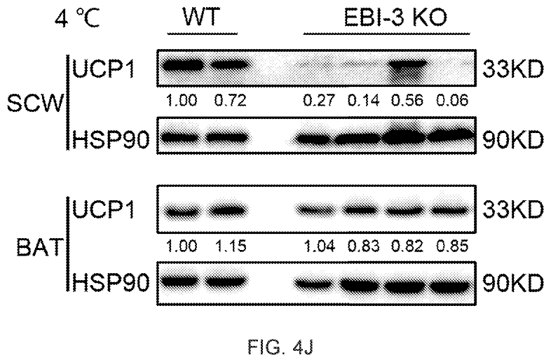

FIG. 4A-FIG. 4J show reduction in energy expenditure and heat generation in IL-27 signal-deficient mice. As for FIG. 4A-FIG. 4C: 8-week-old IL-27Rα KO and WT mice were placed in metabolic cages. Food intake (FIG. 4A), oxygen consumption (FIG. 4B) and energy expenditure (FIG. 4C) (n=7-8) were shown. As for FIG. 4D: BAT and SCW tissue was separated from 6-8-week-old WT-ND and IL-27Rα KO and cut into small pieces (about 0.003 g BAT and about 0.004 g SCW), and a basal oxygen consumption rate (n=5/group, 4-5 pieces/group/mouse) was detected using a Seahorse XF analyzer. As for FIG. 4E: a survival curve of response of IL-27Rα KO and WT mice fed with ND to cold stimulation (4° C., n=9-11) was shown. As for FIG. 4F: an influence of cold stimulation on rectal temperatures of the mice fed with ND (4° C., n=9-11) was shown. As for FIG. 4G: expression of genes of IL-27Rα KO and WT mice fed with a normal diet in SCW was detected using real-time PCR (n=9-11). As for FIG. 4H-FIG. 4J: EBI-3 KO mice and a WT control group fed with a normal diet were recorded at 4° C. cold stimulation with survival curves (FIG. 4H) and rectal temperatures (FIG. 4I) (n=5-6) shown in the drawing. BAT and SCW were collected 12 hours after cold stimulation, lysed and used for immunoblot analysis of UCP1 (FIG. 4J). A band density was quantified with ImageJ, and a UCP1/HSP90 ratio was normalized. The experiment was performed twice with similar results. Data was a mean of biologically independent samples ±s.e.m. An unpaired t-test (FIG. 4D and FIG. 4G) was shown; two-way analysis of variance (FIG. 4A-FIG. 4C, FIG. 4F and FIG. 4I) was shown; and a log rank test (FIG. 4E and FIG. 4H) was shown. *p<0.05, **p<0.01, and **p<0.001.

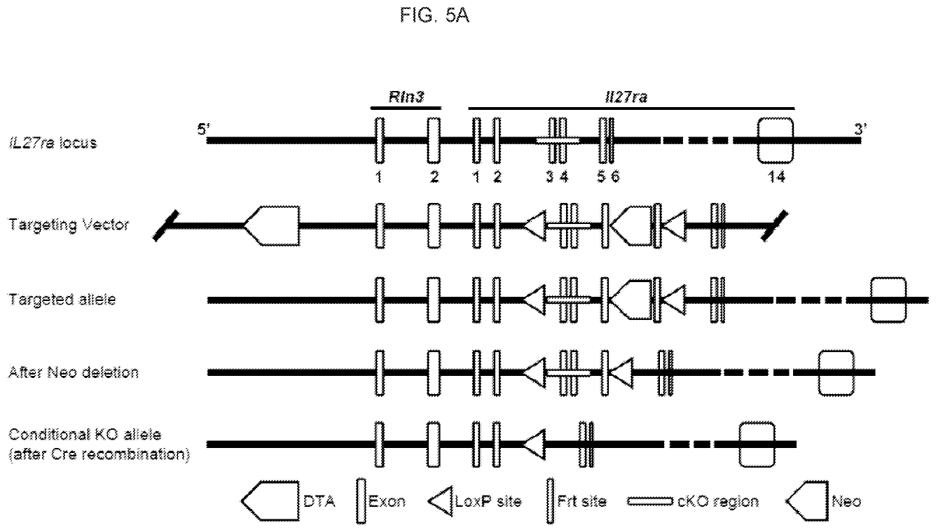

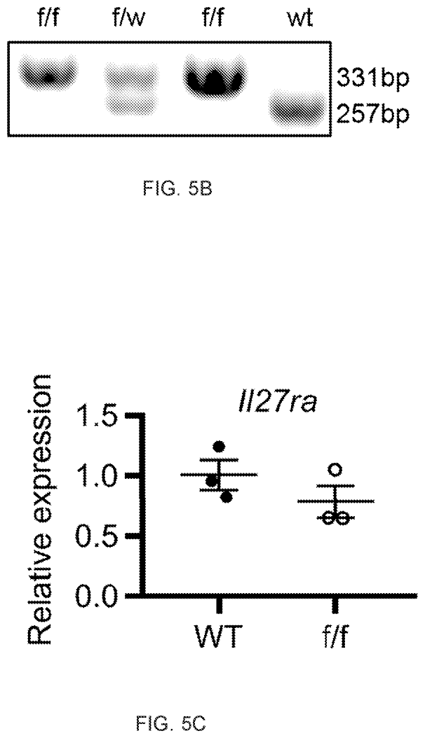

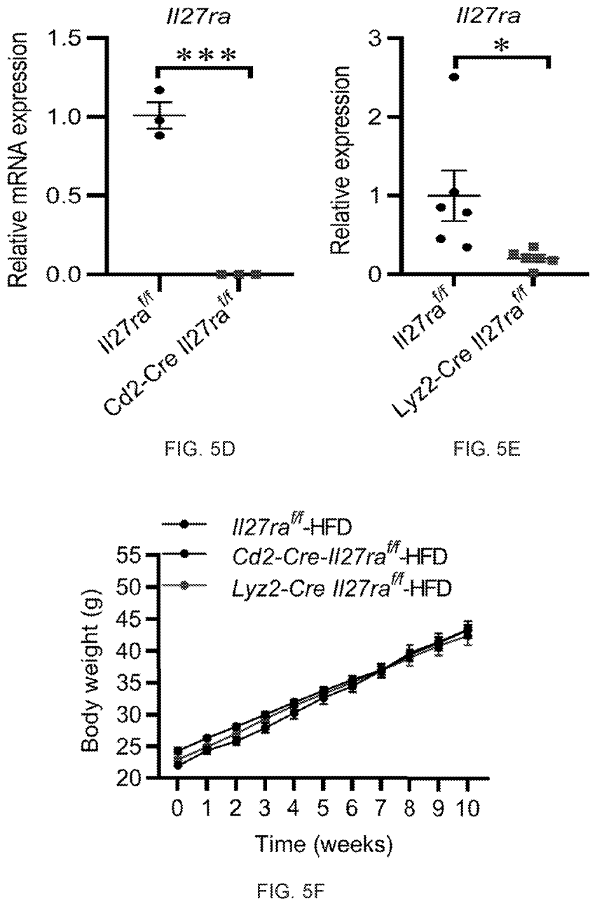

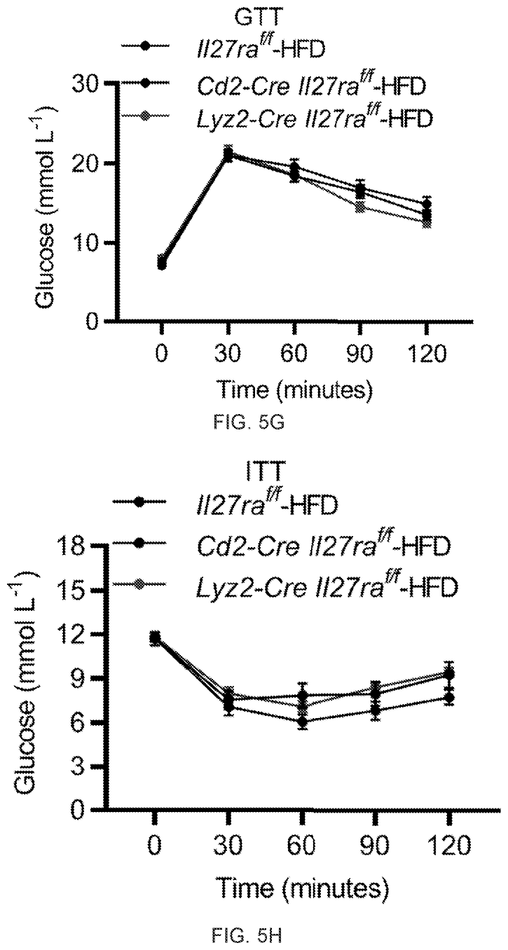

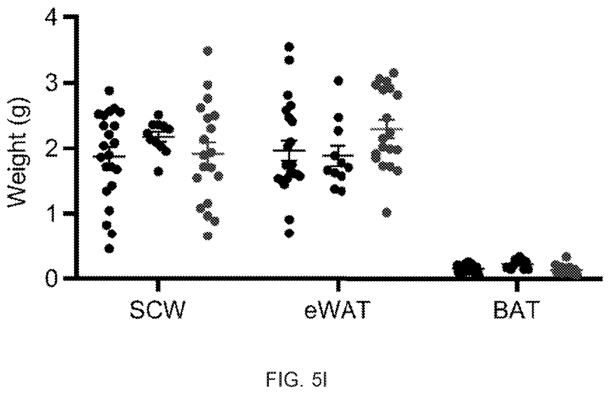

FIG. 5A-FIG. 5I show that thermogenesis of IL-27Rα signals was not achieved by direct action on CD2+ lymphocytes or Lyz2+ myeloid cells. As for FIG. 5A: a schematic model generated for Il27ra^{flox/flox} mice was shown. An Il27ra site (top) was targeted by a targeting vector (second), and the targeting vector contained a homologous sequences of Il27ra including two LoxP sites on two sides of exons 3 and 4 and a Neo selection box. The linearized vector was then delivered to embryonic stem cells by electroporation (C57BL/6), followed by drug selection, PCR screening and Southern blot for confirmation. Homologous recombination produced a floxed allele (Third). After correct targeting of ES clones was confirmed by the Southern blot, selected clones were used for blastocyst microinjection to generate generation F0. It was identified that after hybridization of F0 with Flp-deleter, F1 was confirmed as a germ line passage to cancel the Neo box (Forth). After Cre recombination, mismatching with a floxed Il27ra allele resulted in deletion of exons 3 and 4 (bottom). As for FIG. 5B: genotyping of Il27rad^{f/f} mice was shown. As for FIG. 5C: expression (n=3) of an Il27ra gene in spleens of Il27ra^{f/f} and WT mice was detected by adopting a real-time fluorescent quantitative PCR method. As for FIG. 5D and FIG. 5E: expression of an Il27ra gene in CD3 T cells in spleens of Il27ra^{f/f} and Cd2-Cre Il27ra^{f/f} mice was detected using real-time fluorescent quantitative PCR (n=3, left). Thioglycollate was injected intraperitoneally into Il27ra^{f/f} and Lyz2-Cre Il27ra^{f/f} mice for 4 days, peritoneal macrophages were then collected, and expression of an Il27ra gene was detected by real-time PCR (n=5-6, right). As for FIG. 5F-FIG. 5I: 8-week-old Il27ra^{f/f}, Cd2-Cre-Il27ra^{f/f} and Lyz2-Cre-Il27ra^{f/f} mice were fed with HFD for 10 weeks. As for FIG. 5F: body weight was recorded weekly (n=11-21). GTT (FIG. 5G) and ITT (FIG. 5H) tests (n=11-19) were performed after HFD treatment for 10 weeks. As for FIG. 5I: after HFD treatment for 10 weeks, adipose tissue was collected and weighed (n=11-21). Data was a mean of biologically independent samples ±s.e.m. An unpaired t-test (FIG. 5C, FIG. 5D, FIG. 5E) was shown; two-way analysis of variance (FIG. 5F-FIG. 5H) was shown; and one-way analysis of variance (FIG. 5I) was shown. *p<0.05, and ***p<0.001.

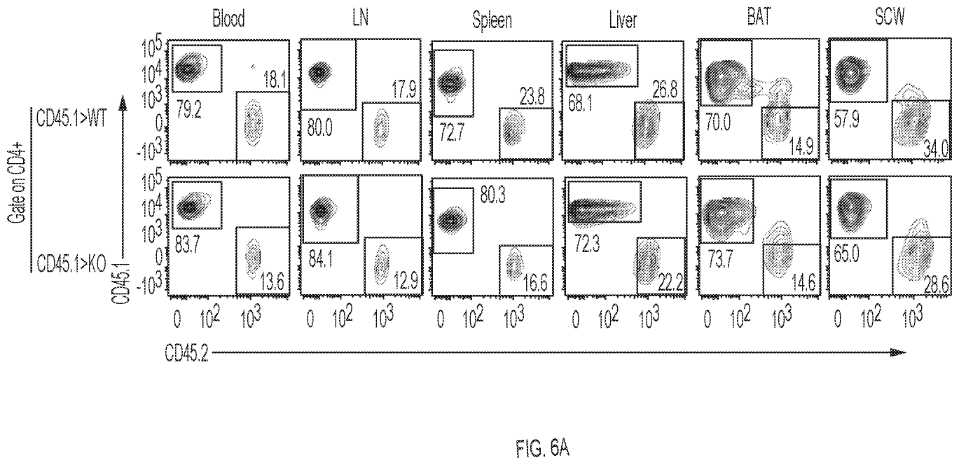

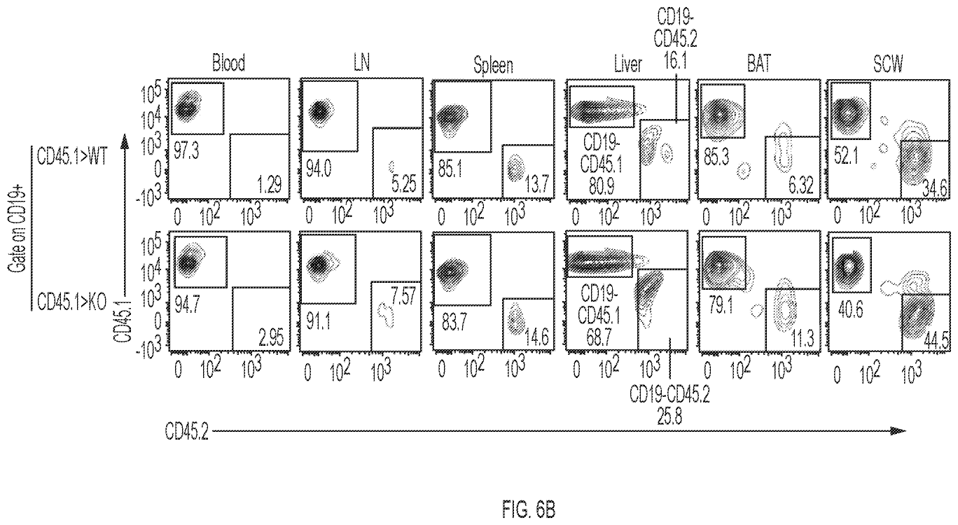

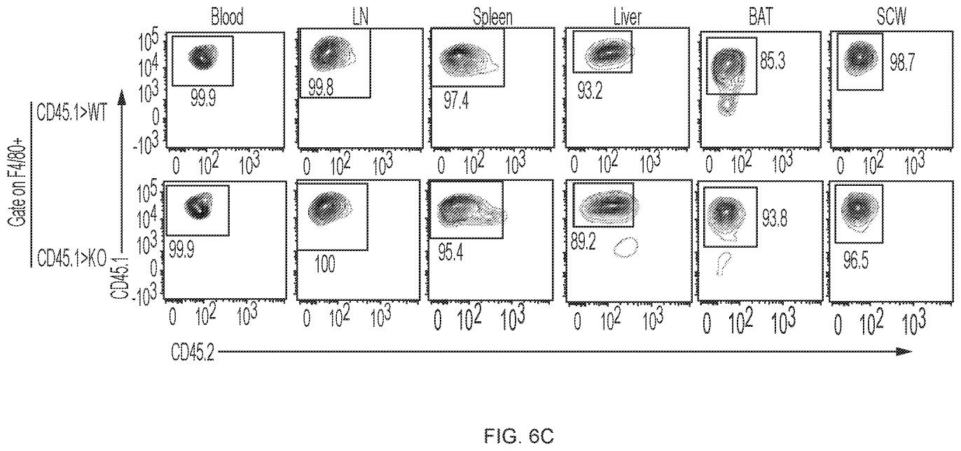

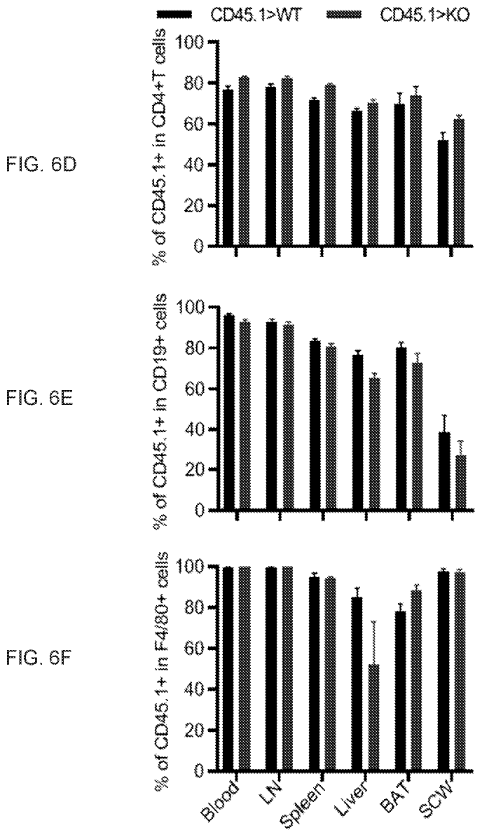

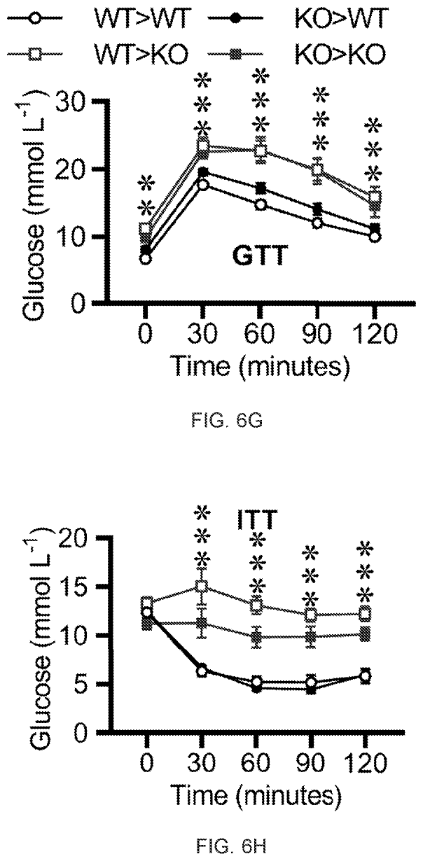

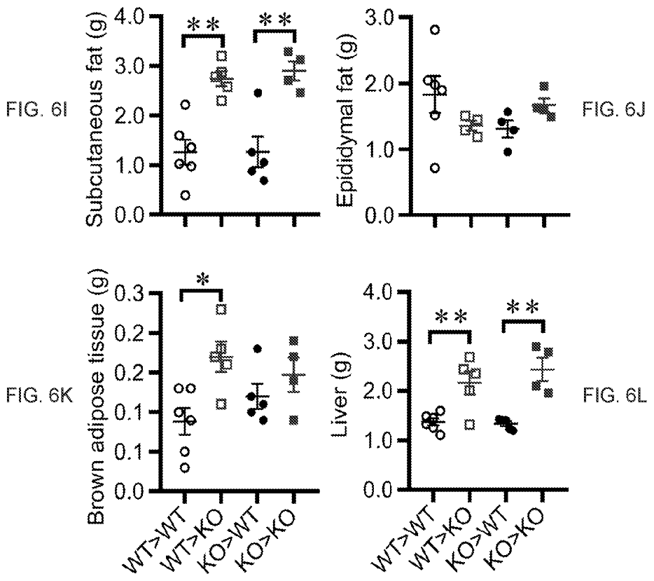

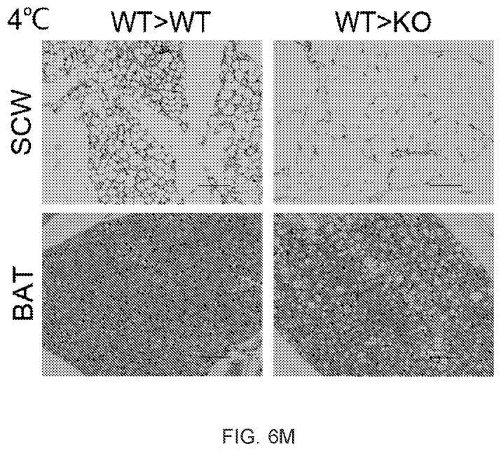

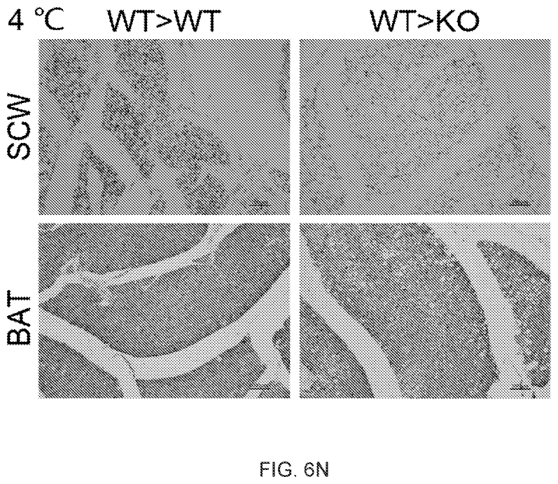

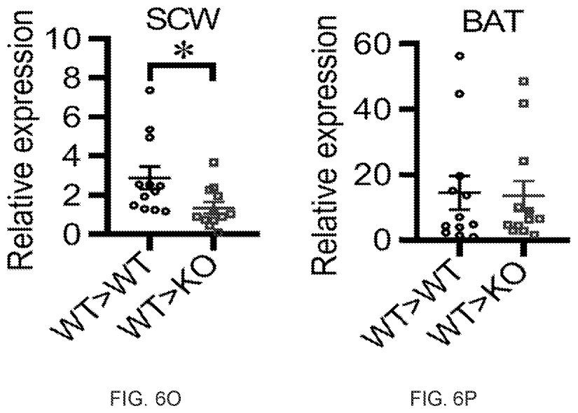

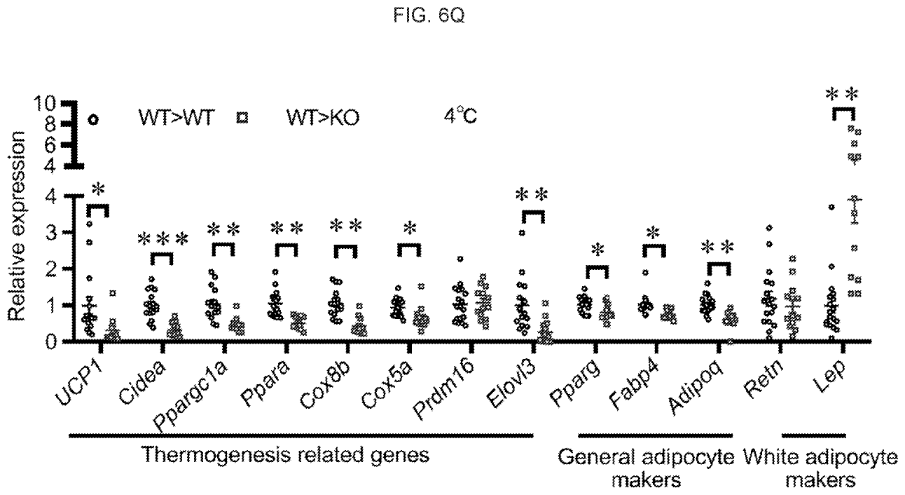

FIG. 6A-FIG. 6Q relate to the phenotype of an IL-27Rα KO and WT chimera in HFD-induced adaptive heat generation. Bone marrow cells from CD45.1 WT strains were transferred to irradiated CD45.2 IL-27Rα KO or CD45.2 WT hosts (1×10⁷ cells/mouse) to generate chimeras. Mice were housed for 8 weeks to reconstitute immune systems. As shown, immune cells were separated from different tissue, and percentages (n=3) of the immune cells in donor (CD45.1+) and host (CD45.2+) cells in CD4+T (top), CD19+B (middle) or F4/80+ macrophages (bottom) were analyzed by a flow cytometer. As for FIG. 6G-FIG. 6Q: bone marrow chimeras were obtained and fed with HFD as shown in a in the drawing. GTT (FIG. 6G, n=10-13) and ITT (FIG. 6H, n=5-7) tests were performed after 10 weeks of HFD treatment. As for FIG. 6I through FIG. 6L: after 10 weeks of HFD treatment, adipose tissue and livers were collected and weighed (n=4-6). As for FIG. 6M-FIG. 6Q: chimera mice fed with a normal diet were subjected to cold stimulation at 4° C. for 12 hours, and SCW and BAT were collected for histological analysis (FIGS. 6M, H and E, scale=100 μm) or UCP1 immunohistochemical staining (FIG. 6N, FIG. 6O, and FIG. 6P, scale=100 μm). Real-time PCR analysis of gene expression of SCW was performed (FIG. 6Q, n=12). Data was a mean of biologically independent samples ±s.e.m. An unpaired t-test (FIG. 6A-FIG. 6F, FIG. 6N-FIG. 6Q) was shown; one-way analysis of variance (FIG. 6Q) was shown; and two-way analysis of variance (FIG. 6G and FIG. 6H) was shown. *p<0.05, **p<0.01, and **p<0.001.

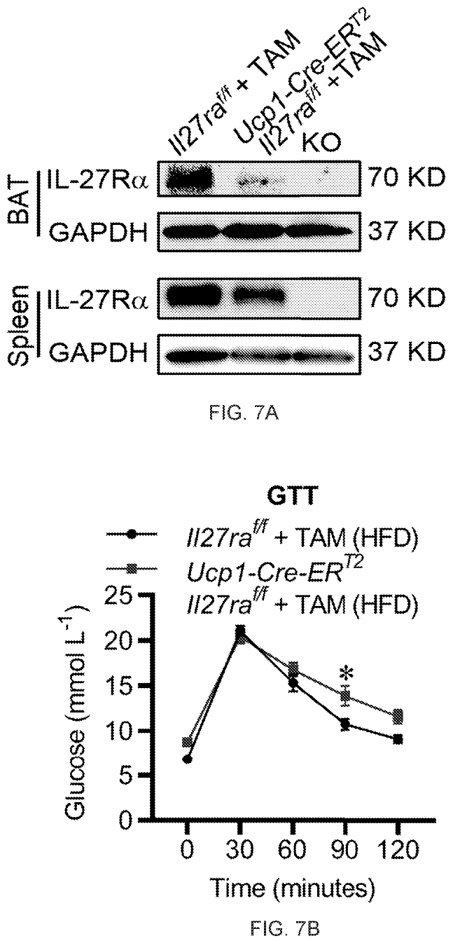

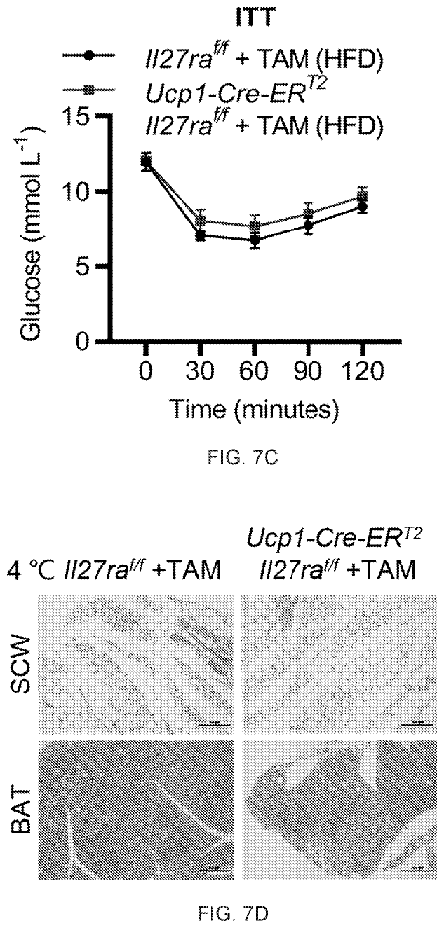

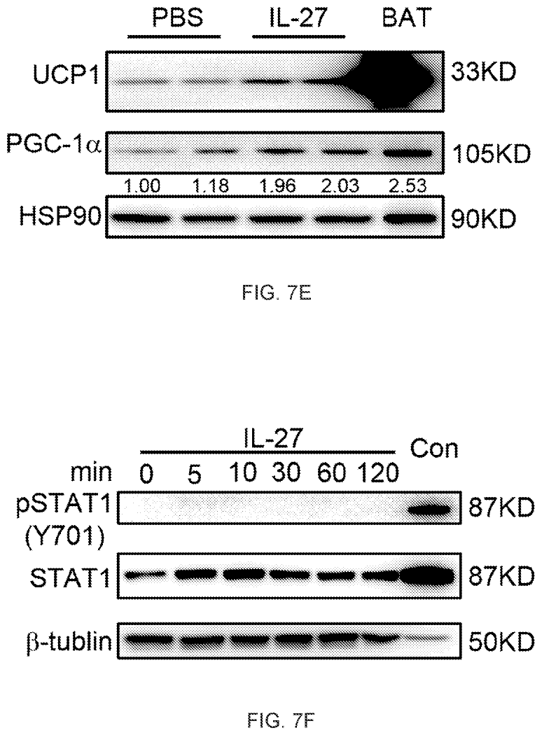

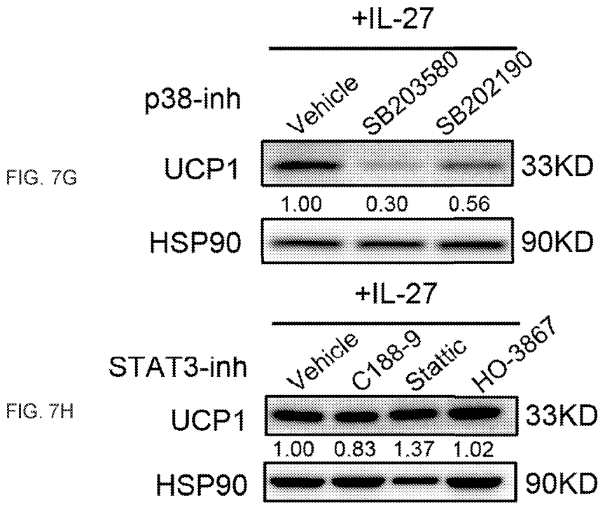

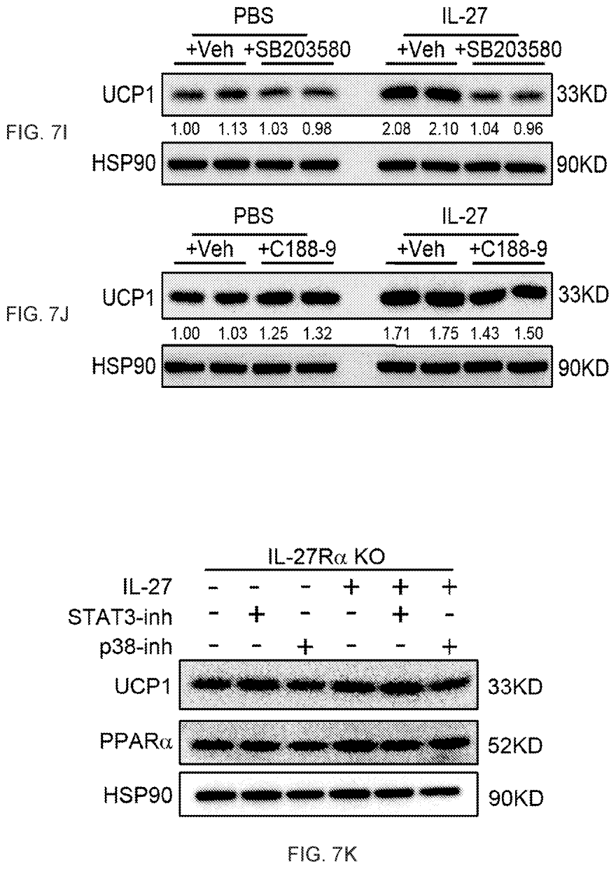

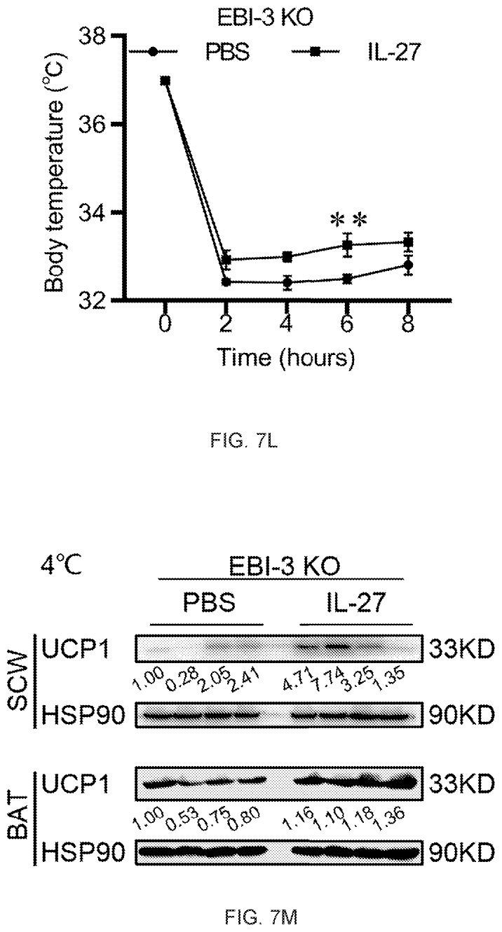

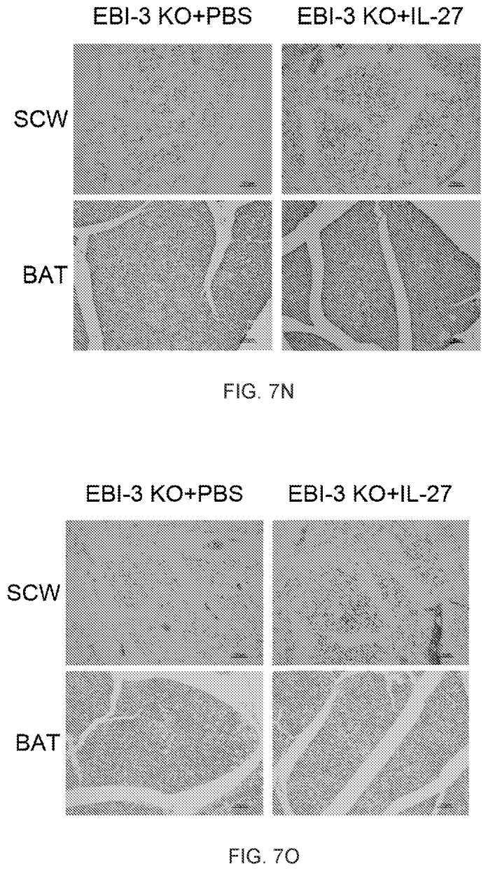

FIG. 7A-FIG. 7O show that IL-27 up-regulates UCP1 and improves browning of subcutaneous white adipose tissue. As for FIG. 7A-FIG. 7C: 8-week-old Il27ra^{f/f} and Ucp1-Cre Il27ra^{f/f} mice were fed with HFD for 10 weeks. After HFD treatment for 10 weeks, GTT (FIG. 7A) and ITT (FIG. 7B) (n=5-6) tests were performed, and adipose tissue and livers were collected and weighed (FIG. 7C, n=5-6). As for FIG. 7D: after Il27ra^{f/f} and Ucp1-Cre Il27ra^{f/f} mice fed with a normal diet were subjected to cold stimulation at 4° C. for 12 hours, SCW and BAT were collected for histological analysis (H and E) with the scale=100 μm. As for FIG. 7E: primary beige adipocytes were generated in vitro from an SVF of WT SCW and then treated with rmIL-27 (100 ng/ml) or PBS for 24 hours. Cells were lysed and a protein extract was used for immunoblot analysis. BAT tissue from WT mice served as positive control. As for FIG. 7F: immunoblot analysis of phosphorylation of STAT1 in the WT primary beige adipocyte extract treated with the rmIL-27 (100 ng/ml) for a specified time was shown. As for FIG. 7G and FIG. 7H: subcutaneous adipocytes of WT mice were cultured in vitro. Two different p38 MAPK inhibitors (SB203580, 10 μM and SB202190, 5 μM) or three STAT3 inhibitors (C188-9, 10 μM; Stattic, 10 μM or HO3867, 20 μM) were added to a medium 0.5 h prior to and during rmIL-27 treatment (100 ng/ml, 12 h). Expression of UCP1 was detected by immunoblot. As for FIG. 7I-FIG. 7J: primary beige adipocytes of SCW from WT (FIG. 7I) or IL-27Rα KO (FIG. 7J) mice were obtained by in vitro culturing. The p38 MAPK inhibitor (SB203580, 10 μM) or STAT3 inhibitor (C188-9, 10 μM) was added to a medium 0.5 h prior to and during rmIL-27 treatment (100 ng/ml, 12 h). Expression of UCP1 or PPARα was analyzed by an immunoblot. As for FIG. 7L-FIG. 7O: EBI-3 KO mice were injected intraperitoneally with rmIL-27 (100 μg/kg) or PBS for 7 consecutive days, and then, cold stimulation was performed at 4° C. As for FIG. 7L: rectal temperatures of the mice for cold stimulation (n=6) were shown. SCW and BAT protein extracts were used for immunoblot analysis after cold stimulation for 24 hours. UCP1 staining (FIG. 7N) and histological analysis (FIG. 7O) (scale=100 μm) were shown. Representative portions were shown. A band density was quantified and normalized with ImageJ. The experiment was repeated twice with similar results. Data was a mean of biologically independent samples ±s.e.m. An unpaired t-test (FIG. 7C) was shown; and two-way analysis of variance (FIG. 7A, FIG. 7B and FIG. 7L) was shown. *p<0.05, **p<0.01, and **p<0.001.

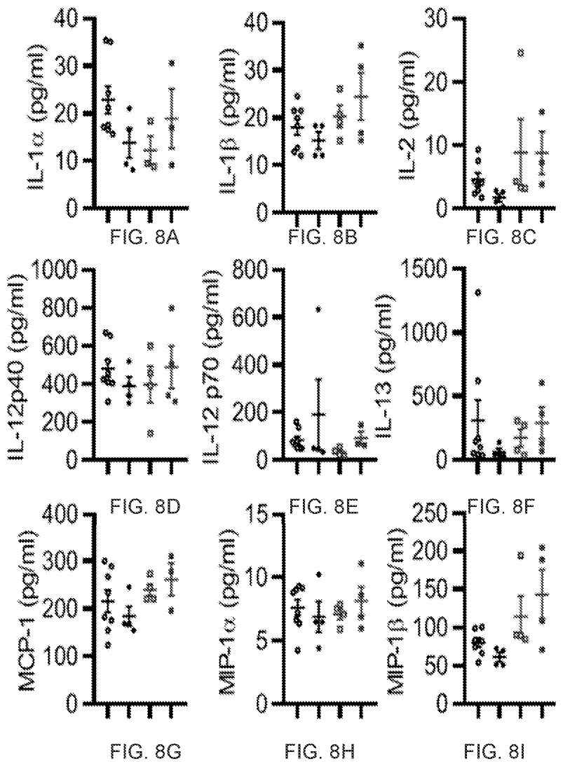

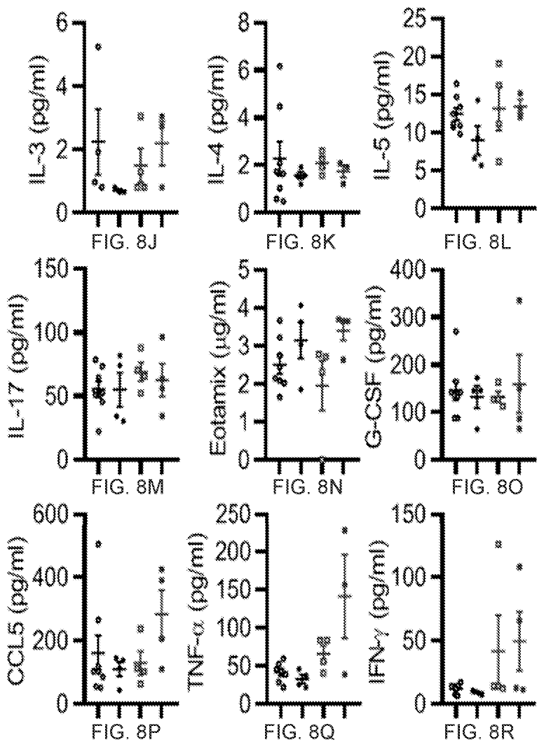

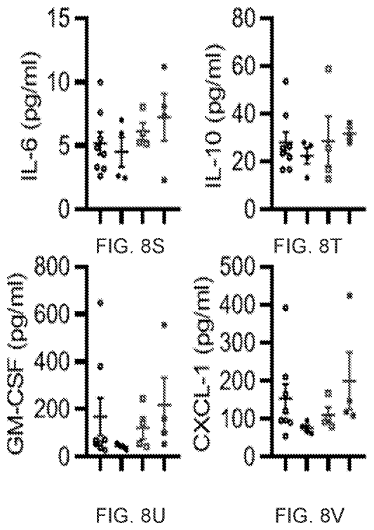

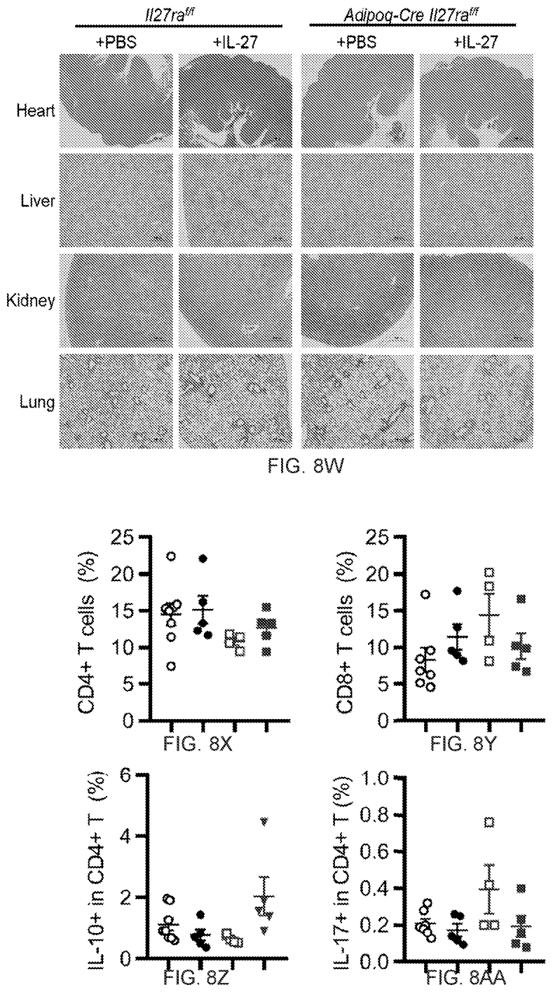

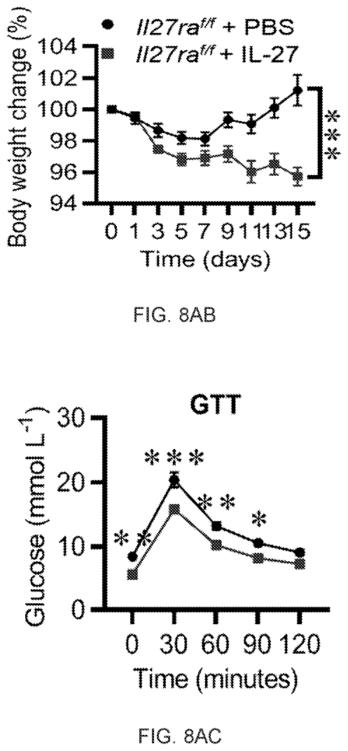

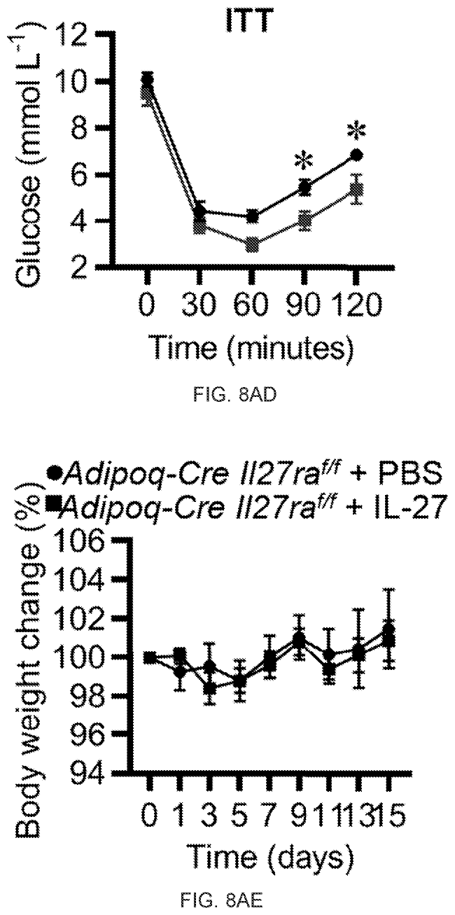

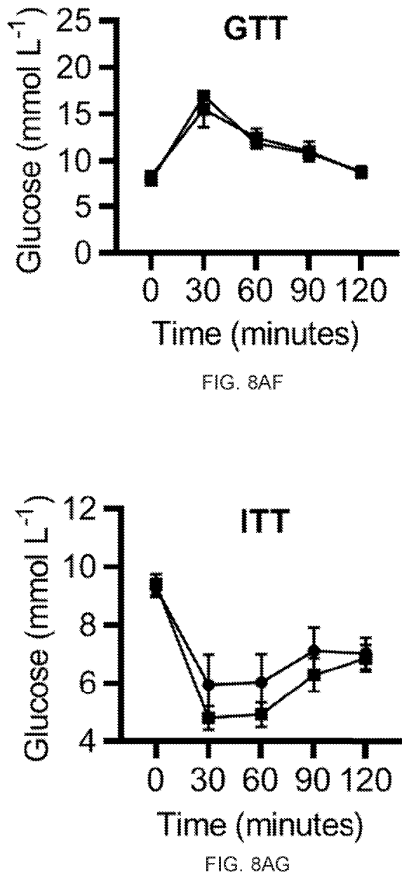

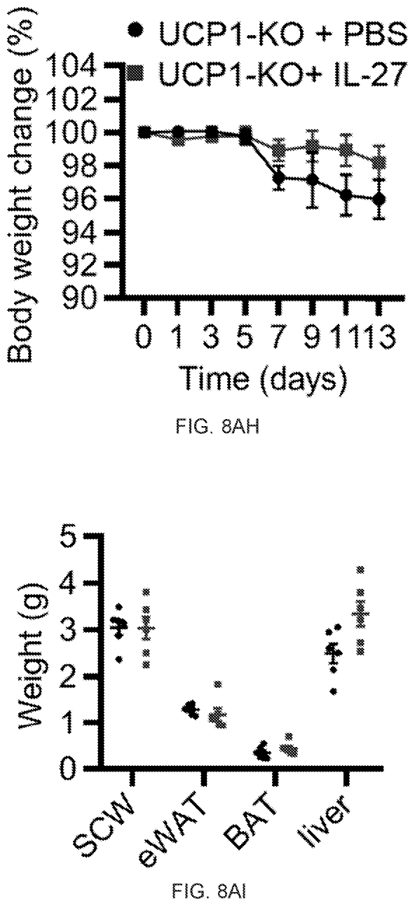

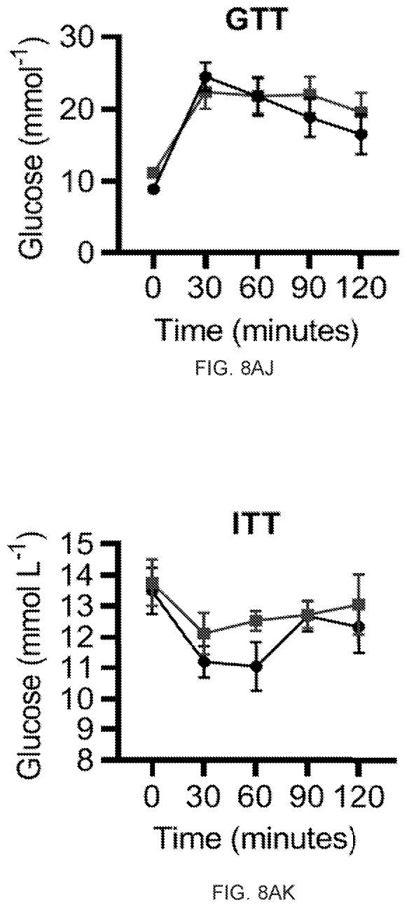

FIG. 8A-FIG. 8AK shows that the effect of IL-27 promoting heat generation. As for FIG. 8A-FIG. 8AG: Il27ra^{f/f} and Adipoq-Cre Il27ra^{f/f} mice were fed with HFD for 32 weeks, and then, intraperitoneal injection of rmIL-27 (100 μg/kg) or PBS was performed every other day for 15 days in total. As for FIG. 8A through FIG. 8V: inflammatory factors in serum was detected by adopting a Bio-Rad Bio-Plex 200 multifunctional analyzer. As for FIG. 8W: representative tissue sections of indicated tissue (H and E) were shown. As for FIG. 8X through FIG. 8AA: a percentage of infiltrated CD4 or CD8 T cells and production of cytokine of hepatic CD4 T cells were analyzed by a flow cytometer. Body weight (FIG. 8AB), GTT (FIG. 8AC) and ITT (FIG. 8AD) (n=5-8) of Il27ra^{f/f} mice were detected and recorded. Body weight (FIG. 8AE), GTT (FIG. 8AF) and ITT (FIG. 8AG) (n=4-5) of Adipoq-Cre Il27ra^{f/f} mice were detected and recorded. As FIG. 8AH-FIG. 8AK: Ucp1-Cre Il27ra^{f/f} mice were fed with HFD for 16 weeks, and then, intraperitoneal injection of rmIL-27 (100 μg/kg) or PBS was performed every other day for 15 days in total. Body weight (FIG. 8AH), tissue weight (FIG. 8AI), a glucose tolerance test (FIG. 8AJ) and an insulin tolerance test (FIG. 8AK) (n=5-6) were measured and recorded. Data was a mean of biologically independent samples ±s.e.m. One-way analysis of variance (FIG. 8A through FIG. 8V and FIG. 8X through FIG. 8AA) and two-way analysis of variance (FIG. 8AB-FIG. 8AH, FIG. 8AJ and FIG. 8AK) were shown; and an unpaired t-test (FIG. 8AI) was shown. *p<0.05, **p<0.01, and **p<0.001.

### 4. Conclusion

Browning of white adipose tissue predisposes mice to increase energy expenditure. In order to examine a systemic metabolic status, in the present disclosure, the energy expenditure and nutrient consumption of the IL-27Rα KO mice and the WT control group having the normal diet or HFD treatment were detected. The energy expenditure of the IL-27Rα KO mice fed with the normal diet was significantly reduced without changing the food intake (shown in FIG. 7A-FIG. 7C). The phenotype was more pronounced when HFD was fed (shown in FIG. 1A-FIG. 1C). The data reveals that IL-27 signals have an effect of maintaining systemic metabolic homeostasis. To determine whether an influence of IL-27Rα deficiency on the energy expenditure may be caused by reduced heat generation, in the present disclosure, an influence of IL-27 signal absence on adaptive heat generation was then examined.

The IL-27Rα KO mice were sensitive to cold-induced hypothermia under the normal diet condition (shown in FIG. 4F and FIG. 4G), and this intolerance was more severe after HFD treatment (shown in FIG. 1D and FIG. 1E). The expression of the key protein (uncoupling protein 1 (UCP1)) regulating thermogenesis in IL-27Rα KO mice was also significantly reduced (shown in FIG. 1F-FIG. 1H). The tissue sections also showed less browning in the brown adipose tissue (BAT) and subcutaneous white adipose tissue (SCW) in the IL-27Rα KO mice (shown in FIG. 1I) and fewer cells containing multilumen lipid droplets (shown in FIG. 1J). The data clearly indicates that the thermogenic activity of the IL-27Rα KO mice is severely impaired. Notably, UCP1 was reduced in the IL-27Rα KO mice prior to cold stimulation (shown in FIG. 1F and FIG. 1H), as was the case with other thermogenic genes (shown in FIG. 4F), which was consistent with reduction in systemic energy expenditure (shown in FIG. 1B and FIG. 1C, and FIG. 4B and FIG. 4C). Furthermore, cold stimulation for the EBI-3 KO mice reproduced the findings in the IL-27Rα KO mice (shown in FIG. 4G-FIG. 84I), which further confirms the thermogenic effect of the IL-27 signals.

Next, the cell types responsible for IL-27 mediated signaling were dissected. First, 4 groups of chimera mice (WT>WT, WT>KO, KO>WT and KO>KO) were generated from the bone marrow of the IL-27Rα KO or WT mice, and these mice were subjected to HFD treatment. Surprisingly, only the chimeras made with IL-27Rα KO (no matter what the donor cells were) were highly susceptible to HFD-induced fat accumulation and cold stimulation (shown in FIG. 2A-FIG. 2C). Compared to wild-type chimeras, thermogenesis of the IL-27Rα KO chimeras was consistently inhibited, which was manifested by a decreased body temperature, decreased multilumen lipid droplets and decreased UCP1 production (shown in FIG. 2A-FIG. 2C). The study indicates that a dominant target of IL-27 is adipocytes instead of immune cells. In fact, IL-27Rα was expressed on mature adipocytes and primary beige adipocytes. Furthermore, heat generation in IL-27Rα^{Δadipo} mice was also inhibited by intolerance to cold stimulation, reduced browning, and reduced UCP1 production (shown in FIG. 2C, FIG. 2E, FIG. 2F and FIG. 2H). In conclusion, the above data indicates that IL-27 promotes heat generation by directly targeting adipocytes.

To investigate the molecular mechanism of action of IL-27 on adipocytes, primary beige adipocytes were directly treated with IL-27 in vitro. The expression of UCP1 was significantly increased after IL-27 treatment, and meanwhile, the expression of the peroxisome proliferator-activated receptor α (PPARα), the peroxisome proliferator-activated receptor γ co-activator 1α (PGC-1α) and the key transcriptional co-activator mediating energy metabolism was up-regulated (shown in FIG. 3A). The typical downstream signal pathways for IL-27Rα in immune cells were JAK/STAT3 and p38 MAPK. Next, whether IL-27 promoted heat generation of the adipocytes by these pathways was examined. Phosphorylation of p38 MAPK was significantly increased under IL-27 stimulation, whereas pSTAT3 was only transiently induced and rapidly regressed (shown in FIG. 3B). It was known that p38 MAPK activates ATF2-mediated transcription of PGC-1α, and PGC-1α is a major regulator of UCP1 and thermogenesis. As expected, IL-27 treatment also enhanced the activation of ATF2 in the primary beige adipocytes, and then, PGC-1α was up-regulated, which could be canceled by pharmacological inhibition of the activity of p38 MAPK (shown in FIG. 3A-FIG. 3C). Furthermore, cells with p38 MAPK inhibition were tolerant to IL-27-induced UCP1 promotion (shown in FIG. 3C). However, the response of up regulation of UCP1 by IL-27 to STAT3 inhibition was essentially unaffected, although it also decreased the expression of PGC-1α (shown in FIG. 3C). These results indicates that IL-27 enhances thermogenesis mainly through the p38 MAPK-PGC-1α signal pathway. Interestingly, inhibition of both p38 and STAT3 failed to cancel the up regulation of PPARα, which suggests that effects of IL-27 may involve additional pathways.

Furthermore, supplementation of the EBI-3 KO mice with IL-27 improved UCP1-mediated heat generation and avoided low temperatures in cold stimulation (FIG. 4A-FIG. 4J).

In summary, the results indicate that the IL-27 signal is indispensable for the full adaptive heat generation. Direct targeting of IL-27 to adipocytes induces activation of p38 MAPK, thereby driving activation of ATF2 and subsequent expression of PGC-1α and UCP1. In conclusion, these findings provides new insights and extends understanding of the mechanism of in vivo heat generation.

While the present disclosure has been described with reference to the exemplary embodiments, it is to be understood that the disclosure is not limited to the disclosed exemplary embodiments. Various modifications or variations may be made to the exemplary embodiments of the present description without departing from the scope or spirit of the present disclosure. The scope of the claims should be based on the broadest interpretation to encompass all modifications and equivalent structures and functions.

### INDUSTRIAL APPLICABILITY

The present disclosure provides the composition and method for controlling heat generation within an organism, and the use, and the present disclosure discloses the functions of the IL-27 signals in adjustment of heat generation. Molecular mechanism research proves that the IL-27 signals directly act on adipocytes, activate p38 MAPK-PGC1 signals and stimulate generation of UCP1. Therapeutic administration of IL-27 reverses hypothermia of EBI-3 KO mice induced at the low temperature. Therefore, the present disclosure reveals a key role of the IL-27 signals in an energy metabolism process, provides a feasible solution for controlling the body temperature, and has a remarkable industrial practical performance and high market value.

What is claimed is:

1. A composition for controlling heat generation within an organism, comprising a molecule for adjusting binding of EBI-3 and p28 or binding of dimers of EBI-3 and p28 and receptors thereof, or a downstream signal pathway.

2. The composition according to claim 1, wherein the adjusting refers to inhibition or reduction of the binding of EBI-3 and p28, or inhibition or reduction of the binding of the dimers of EBI-3 and p28 and the receptors thereof, or down regulation of the downstream signal pathway, and the controlling refers to inhibition of the heat generation, thereby reducing a body temperature of the organism.

3. The composition according to claim 2, wherein the molecule comprises a biological macromolecule or small molecule compound, comprising an anti-EBI-3 antibody, an anti-p28 antibody, an anti-IL-27R antibody, an EBI-3 soluble fragment, a p28 soluble fragment and an IL-27R soluble fragment.

4. The composition according to claim 1, wherein the adjusting refers to promotion or enhancement of the binding of EBI-3 and p28, or promotion or enhancement of the binding of the dimers of EBI-3 and p28 and the receptors thereof, or up regulation of the downstream signal pathway, and the controlling refers to promotion of the heat generation, thereby improving a body temperature of the organism.

5. The composition according to claim 4, wherein the molecule comprises a recombinant IL-27 protein, a nuclear acid fragment like mRNA, virus or plasmid DNA that can express IL-27 protein in vivo, a modifier thereof, and a polypeptide or a small molecule compound which can activate activity of IL-27R.

6. A method for controlling heat generation within an organism, wherein a molecule for adjusting binding of EBI-3 and p28 or binding of dimers of EBI-3 and p28 and receptors thereof, or a downstream signal pathway is applied to the organism.

7. The method according to claim 6, wherein the organism comprises a human subject or tissue or cell thereof, a mammal or tissue or cell thereof, or a microorganism.

8. The method according to claim 7, wherein the tissue or cell is in vitro tissue or cell.

9. A method for screening a candidate compound capable of controlling heat generation within an organism, comprising a step of measuring a binding condition of EBI-3 and p28, or a binding condition of dimers of EBI-3 and p28 and receptors thereof, or a downstream signal pathway triggered by the binding before and after application of a to-be-tested compound.

10. The method according to claim 9, wherein when the binding of EBI-3 and p28 is inhibited or reduced or the binding of the dimers of EBI-3 and p28 and the receptors thereof is inhibited or reduced or a downstream signal triggered by the binding is down-regulated after the to-be-tested compound is applied, the to-be-tested compound is identified as a candidate compound for inhibiting heat generation within the organism.

11. The method according to claim 9, wherein when the binding of EBI-3 and p28 is promoted or enhanced or the binding of the dimers of EBI-3 and p28 and the receptors thereof is promoted or enhanced or the downstream signal triggered by the binding is up-regulated after the to-be-tested compound is applied, the to-be-tested compound is identified as a candidate compound for promoting heat generation within the organism.

12. The method according to claim 9, comprising:
(a) measuring a parameter of the binding of EBI-3 and p28 or a parameter of the binding of the dimers of EBI-3 and p28 and the receptors thereof, or a parameter of an activation condition of the downstream signal pathway in an organism model, so as to obtain a first measured value;
(b) after the to-be-tested compound is applied to the organism model, measuring the parameter of the binding of EBI-3 and p28 or the parameter of the binding of the dimers of EBI-3 and p28 and the receptors thereof, or the parameter of the activation condition of the downstream signal pathway in the organism model, so as to obtain a second measured value; and
(c) comparing the first measured value and the second measured value.

13. The method according to claim 9, comprising:
(a) measuring a parameter of the binding of EBI-3 and p28 or a parameter of the binding of the dimers of EBI-3 and p28 and the receptors thereof, or a parameter of an activation condition of the downstream signal pathway in a first organism model, so as to obtain a first measured value;
(b) measuring a parameter of the binding of EBI-3 and p28 or a parameter of the binding of the dimers of EBI-3 and p28 and the receptors thereof, or a parameter of an activation condition of the downstream signal pathway in a second organism model, so as to obtain a second measured value, wherein the first organism model and the second organism model belong to the same model, the to-be-tested compound is not applied to the first organism model, and the to-be-tested compound is applied to the second organism model; and
(c) comparing the first measured value and the second measured value.

14. The method according to claim 9, comprising:
(a) measuring a parameter of the binding of EBI-3 and p28 or a parameter of the binding of the dimers of EBI-3 and p28 and the receptors thereof, or a parameter of an activation condition of the downstream signal pathway in an organism model after the to-be-tested compound is applied, so as to obtain a measured value; and
(b) comparing the measured value with a reference value, wherein the reference value is a parameter of the binding of EBI-3 and p28 or a parameter of the binding of the dimers of EBI-3 and p28 and the receptors thereof, or a parameter of an activation condition of the downstream signal pathway measured by the organism model when the to-be-tested compound is not applied.

15. The method according to claim 12, wherein the organism model comprises an animal model or a cell model.

16. (canceled)

17. (canceled)

18. The composition according to claim 2, wherein the adjusting refers to promotion or enhancement of the binding of EBI-3 and p28, or promotion or enhancement of the binding of the dimers of EBI-3 and p28 and the receptors thereof, or up regulation of the downstream signal pathway, and the controlling refers to promotion of the heat generation, thereby improving a body temperature of the organism.

19. The method according to claim 10, wherein when the binding of EBI-3 and p28 is promoted or enhanced or the binding of the dimers of EBI-3 and p28 and the receptors thereof is promoted or enhanced or the downstream signal triggered by the binding is up-regulated after the to-be-tested compound is applied, the to-be-tested compound is identified as a candidate compound for promoting heat generation within the organism.

20. The method according to claim 10, comprising:
(a) measuring a parameter of the binding of EBI-3 and p28 or a parameter of the binding of the dimers of EBI-3 and p28 and the receptors thereof, or a parameter of an activation condition of the downstream signal pathway in an organism model, so as to obtain a first measured value;
(b) after the to-be-tested compound is applied to the organism model, measuring the parameter of the binding of EBI-3 and p28 or the parameter of the binding of the dimers of EBI-3 and p28 and the receptors thereof, or the parameter of the activation condition of the downstream signal pathway in the organism model, so as to obtain a second measured value; and
(c) comparing the first measured value and the second measured value.

21. The method according to claim 10, comprising:
(a) measuring a parameter of the binding of EBI-3 and p28 or a parameter of the binding of the dimers of EBI-3 and p28 and the receptors thereof, or a parameter of an activation condition of the downstream signal pathway in a first organism model, so as to obtain a first measured value;
(b) measuring a parameter of the binding of EBI-3 and p28 or a parameter of the binding of the dimers of EBI-3 and p28 and the receptors thereof, or a parameter of an activation condition of the downstream signal pathway in a second organism model, so as to obtain a second measured value, wherein the first organism model and the second organism model belong to the same model, the to-be-tested compound is not applied to the first organism model, and the to-be-tested compound is applied to the second organism model; and
(c) comparing the first measured value and the second measured value.

22. The method according to claim 10, comprising:
(a) measuring a parameter of the binding of EBI-3 and p28 or a parameter of the binding of the dimers of EBI-3 and p28 and the receptors thereof, or a parameter of an activation condition of the downstream signal pathway in an organism model after the to-be-tested compound is applied, so as to obtain a measured value; and
(b) comparing the measured value with a reference value, wherein the reference value is a parameter of the binding of EBI-3 and p28 or a parameter of the binding of the dimers of EBI-3 and p28 and the receptors thereof, or a parameter of an activation condition of the downstream signal pathway measured by the organism model when the to-be-tested compound is not applied.


SEQUENCE LISTING (30 sequences)

SEQ ID NO: 1 - DNA, 19 bp, synthetic construct
  source 1..19: mol_type = other DNA; note = primer
tcattatgccgaggatttg

SEQ ID NO: 2 - DNA, 18 bp, synthetic construct
  source 1..18: mol_type = other DNA; note = primer
gcctcccatctccttcat

SEQ ID NO: 3 - DNA, 20 bp, synthetic construct
  source 1..20: mol_type = other DNA; note = primer
ctactgaacttcggggtgat

SEQ ID NO: 4 - DNA, 19 bp, synthetic construct
  source 1..19: mol_type = other DNA; note = primer
caggcttgtcactcgaatt

SEQ ID NO: 5 - DNA, 20 bp, synthetic construct
  source 1..20: mol_type = other DNA; note = primer
tccagttgccttcttgggac

SEQ ID NO: 6 - DNA, 21 bp, synthetic construct
  source 1..21: mol_type = other DNA; note = primer
gtgtaattaagcgccgacttg

SEQ ID NO: 7 - DNA, 27 bp, synthetic construct
  source 1..27: mol_type = other DNA; note = primer
ggctggtgcaaagaaacatggacttga

SEQ ID NO: 8 - DNA, 25 bp, synthetic construct
  source 1..25: mol_type = other DNA; note = primer
tgcagacagagacgccattccacat

SEQ ID NO: 9 - DNA, 25 bp, synthetic construct
  source 1..25: mol_type = other DNA; note = primer
caaccaacaagtgatattctccatg

SEQ ID NO: 10 - DNA, 21 bp, synthetic construct
  source 1..21: mol_type = other DNA; note = primer
gatccacactctccagctgca

SEQ ID NO: 11 - DNA, 21 bp, synthetic construct
  source 1..21: mol_type = other DNA; note = primer
gaaaactccatgcttgaagaa

SEQ ID NO: 12 - DNA, 21 bp, synthetic construct
  source 1..21: mol_type = other DNA; note = primer
tctttcagtgatgtggacttg

SEQ ID NO: 13 - DNA, 20 bp, synthetic construct
  source 1..20: mol_type = other DNA; note = primer
tcaccgagctctgttgacaa

SEQ ID NO: 14 - DNA, 20 bp, synthetic construct
  source 1..20: mol_type = other DNA; note = primer
ccacacttctctttttggcg

SEQ ID NO: 15 - DNA, 21 bp, synthetic construct
  source 1..21: mol_type = other DNA; note = primer
tgagcaacatcacacaagacc

SEQ ID NO: 16 - DNA, 19 bp, synthetic construct
  source 1..19: mol_type = other DNA; note = primer
ggccttgcggttacagagg

SEQ ID NO: 17 - DNA, 21 bp, synthetic construct
  source 1..21: mol_type = other DNA; note = primer
actgccacacctccagtcatt

SEQ ID NO: 18 - DNA, 21 bp, synthetic construct
  source 1..21: mol_type = other DNA; note = primer
ctttgcctcactcaggattgg

SEQ ID NO: 19 - DNA, 20 bp, synthetic construct
  source 1..20: mol_type = other DNA; note = primer
gaaccatgaagccaacgact

SEQ ID NO: 20 - DNA, 20 bp, synthetic construct
  source 1..20: mol_type = other DNA; note = primer
gcgaagttcacagtggttcc

SEQ ID NO: 21 - DNA, 21 bp, synthetic construct
  source 1..21: mol_type = other DNA; note = primer
tgctcttctgtatcgcccagt

SEQ ID NO: 22 - DNA, 21 bp, synthetic construct
  source 1..21: mol_type = other DNA; note = primer
gccgtgttaaggaatctgctg

SEQ ID NO: 23 - DNA, 19 bp, synthetic construct
  source 1..19: mol_type = other DNA; note = primer
cagcacggtgaagccattc

SEQ ID NO: 24 - DNA, 17 bp, synthetic construct
  source 1..17: mol_type = other DNA; note = primer
gcgtgcatccgcttgtg

SEQ ID NO: 25 - DNA, 20 bp, synthetic construct
  source 1..20: mol_type = other DNA; note = primer
gaatcattatgacggcagca

SEQ ID NO: 26 - DNA, 22 bp, synthetic construct
  source 1..22: mol_type = other DNA; note = primer
tcatgtacaccgtgatgtggta

SEQ ID NO: 27 - DNA, 21 bp, synthetic construct
  source 1..21: mol_type = other DNA; note = primer
tccgcgttctcatgtaggtct

SEQ ID NO: 28 - DNA, 21 bp, synthetic construct
  source 1..21: mol_type = other DNA; note = primer
ggacctgatgcaaccctatga

SEQ ID NO: 29 - DNA, 24 bp, synthetic construct
  source 1..24: mol_type = other DNA; note = primer
ctggttctggtatggtttggggtt

SEQ ID NO: 30 - DNA, 24 bp, synthetic construct
  source 1..24: mol_type = other DNA; note = primer
tgaaagaactcaacagtgggccgg